(19) United States
(12) Patent Application Publication
(10) Pub. No.: US 2024/0307480 A1
(43) Pub. Date: Sep. 19, 2024

(54) MELFLUFEN FOR USE IN THE TREATMENT OF MULTIPLE MYELOMA

(71) Applicant: Oncopeptides AB, Stockholm (SE)

(72) Inventor: Jakob LINDBERG, Stockholm (SE)

(73) Assignee: Oncopeptides AB, Stockholm (SE)

(21) Appl. No.: 18/576,878

(22) PCT Filed: Jul. 7, 2022
(86) PCT No.: PCT/EP2022/068974

§ 371 Date: Jan. 5, 2024

(30) Foreign Application Priority Data
Jul. 8, 2021 (GB) 2109894.2
Sep. 10, 2021 (GB) 2112976.2

(51) Int. Cl. A61K 38/05; A61K 31/216; A61K 31/573; A61K 45/06; A61P 35/00

(52) U.S. Cl. CPC A61K 38/05; A61K 31/216; A61K 31/573; A61K 45/06; A61P 35/00

(57) ABSTRACT

The present invention relates to a particularly advantageous new therapeutic use of melflufen (melphalan flufenamide; L-melphalanyl-4-fluoro-L-phenylalanine ethyl ester), or a salt thereof: in the treatment or prophylaxis of multiple myeloma in a patient having multiple myeloma who:—has not received a stem cell transplant; or—has received a stem cell transplant that was at least 5 years ago; or—is 75 years old or older; or—has not received a stem cell transplant and is 75 years old or older, or has received a stem cell transplant that was at least 5 years ago and is 75 years old or older; or—has received a stem cell transplant and the disease subsequently progressed at least 36 months after the transplant. The inventors have found that melflufen, and in particular melflufen in combination with dexamethasone, is surprisingly effective for the treatment or prophylaxis of multiple myeloma in these patients. More particularly, the present inventors have found that melflufen demonstrates superior anti-neoplastic activity in comparison to other treatments for multiple myeloma in these patient populations.

### FIELD OF THE INVENTION

The present invention relates to a particularly advantageous new therapeutic use of melflufen (melphalan flufenamide; L-melphalanyl-4-fluoro-L-phenylalanine ethyl ester), or a salt thereof, for use in the treatment or prophylaxis of multiple myeloma in a patient having multiple myeloma who
has not received a stem cell transplant; or
has received a stem cell transplant that was at least 5 years ago; or
is 75 years old or older; or
has not received a stem cell transplant and is 75 years old or older, or has received a stem cell transplant that was at least 5 years ago and is 75 years old or older; or
has received a stem cell transplant and the disease subsequently progressed at least 36 months after the transplant.

### BACKGROUND OF THE INVENTION

Multiple myeloma (MM) is a malignant cancer of the differentiated plasma cells. It is characterized by clonal proliferation of plasma cells in the bone marrow and the production of excessive amounts of a monoclonal immunoglobulin (usually of the IgG or IgA type or free urinary light chain [paraprotein, M-protein or M-component]). Relapsed-refractory multiple myeloma (RRMM) is a specific sub-type of multiple myeloma, and can be defined as multiple myeloma that initially responds to treatment, but does not respond to treatment after relapse.

MM is the second most common hematologic malignancy and nearly 24,000 patients with myeloma are diagnosed in the United States each year. Patients with MM may experience significant detriment to quality of life, including bone pain, bone fractures, fatigue, anaemia, infections, hypercalcemia, hyperviscosity and renal function compromise (including renal failure). The disease course for MM varies with the disease stage at diagnosis, cytogenetic profile, as well as age and patient comorbidities. The disease is ultimately fatal, with a median survival of approximately 3 to 5 years and a 5-year survival estimated at 44.9% (“Surveillance, Epidemiology, and End Results Program Cancer statistics Stat Fact Sheets: Myeloma.” National Cancer Institute; http://www.seer.cancer.gov/statfacts/html/mulmy.html). However, some patients can live longer than 10 years.

In many countries, the standard of care for fit multiple myeloma patients is to receive (after completion of induction therapy) high-dose chemotherapy (HDT) with autologous stem cell rescue. Autologous stem cell rescue is often referred to as autologous stem cell transplant (ASCT). Autologous stem cell transplant can provide significant remission that is both long and deep, extending survival.

The treatment options for patients who are not able to undergo autologous stem cell transplant are limited. Particularly in the relapsed/refractory setting, the results of phase 3 randomized studies may not always be applicable to the transplant-ineligible population, necessitating extrapolation of data obtained from a predominantly younger/transplant eligible population, with possibly different disease biology and toxicity profiles. A review of treatment options for transplant-ineligible multiple myeloma patients in 2021 (Elnair and Holstein, Oncology, Vol 35, Issue 4,Pages: 170-182) concluded that the best answer for those patients is enrolment in a clinical trial and consultation with a MM specialist and geriatric oncologist. That is to say that no approved treatment is reliably beneficial in this patient group.

A Cochrane Review published in 2019 (“Multiple drug combinations of bortezomib, lenalidomide, and thalidomide for first-line treatment in adults with transplant-ineligible multiple myeloma: a network meta-analysis”. Piechotta V, et al, Cochrane Database of Systematic Reviews 2019, Issue 11. Art. No.: CD013487. DOI: 10.1002/14651858.CD013487.) concluded that VRDc (continuous therapy with bortezomib, lenalidomide and dexamethasone) showed the highest overall survival benefits, compared to MP (melphalan and prednisone). RD (lenalidomide and dexamethasone) and TMP (thalidomide, melphalan and prednisone) also improved overall survival compared to MP. However, these combinations of drugs also led to more adverse events compared to MP, and led to more people stopping treatment. The authors thus concluded that more trials are needed that look carefully at both harms and quality of life.

Melflufen (also known as melphalan flufenamide and L-melphalanyl-4-fluoro-L-phenylalanine ethyl ester), is an anti-tumor agent useful in treatment of multiple myeloma. Melflufen is described in WO 01/96367 and WO 2014/065751. The structure of the hydrochloride salt of melflufen is shown in Scheme 1 below:

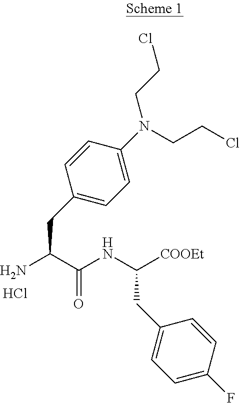 [SMILES: Cl ; CCOC(=O)[C@H](Cc1ccc(F)cc1)NC(=O)[C@@H](N)Cc1ccc(N(CCCl)CCCl)cc1]

Melflufen is a potent and highly lipophilic alkylating agent and it achieves targeted delivery of alkylating metabolites to tumor cells. Due to its high lipophilicity, melflufen rapidly enters tumor cells where it is immediately cleaved by peptidases leading to entrapment and enrichment of alkylating payload (Stiller CA, et al. Cancer Epidemiol. 2018;56:146-153). Overexpression of peptidases is often seen in tumor cells and this might be responsible for high melflufen sensitivity (Stiller CA, et al. Cancer Epidemiol. 2018;56:146-153). Esterases may also play a role in this observed effect. Results of trials of melflufen in human MM sufferers have been published and show clinically meaningful efficacy and a manageable safety profile in patients with heavily pretreated RRMM, including those with triple-class-refractory and extramedullary disease (Richardson, P. G., et al, Journal of Clinical Oncology 2021 39:7, 757-767). The effectiveness of melflufen in patients who are not eligible for transplant has not been studied or reported.

Thus, whilst recent improvements in therapies for MM have significantly prolonged survival for patients who are eligible for autologous stem cell transplant, the outcomes of those same treatments in patients who are not able to undergo autologous stem cell transplant are highly uncertain and significantly less favourable, especially so in relapsed or refractory patients. There thus remains an intense need for further treatment options in MM patients who are not able to undergo autologous stem cell transplant. There also remains an intense need for further treatment options in MM patients who have undergone autologous stem cell transplant in the past (for example at least 5 years in the past) but are not eligible for further autologous stem cell transplant, and especially so in relapsed or refractory patients.

### SUMMARY OF THE INVENTION

The present invention provides melflufen, or a salt(s) thereof, for use in the treatment or prophylaxis of multiple myeloma in a patient having multiple myeloma who
has not received a stem cell transplant; or
has received a stem cell transplant that was at least 5 years ago; or
is 75 years old or older; or
has not received a stem cell transplant and is 75 years old or older, or has received a stem cell transplant at least 5 years ago and is 75 years old or older; or
has received a stem cell transplant and the disease subsequently progressed at least 36 months after the transplant.

The present invention further provides a method for the treatment or prophylaxis of multiple myeloma, comprising the step of administering melflufen, or a salt thereof, to a patient having multiple myeloma who
has not received a stem cell transplant; or
has received a stem cell transplant that was at least 5 years ago; or
is 75 years old or older; or
has not received a stem cell transplant and is 75 years old or older, or has received a stem cell transplant that was at least 5 years ago and is 75 years old or older; or
has received a stem cell transplant and the disease subsequently progressed at least 36 months after the transplant.

The present invention further provides a pharmaceutical formulation comprising melflufen, or a salt thereof, for use in the treatment and/or prophylaxis of multiple myeloma in a patient having multiple myeloma who
has not received a stem cell transplant; or
has received a stem cell transplant that was at least 5 years ago; or
is 75 years old or older; or
has not received a stem cell transplant and is 75 years old or older, or has received a stem cell transplant that was at least 5 years ago and is 75 years old or older; or
has received a stem cell transplant and the disease subsequently progressed at least 36 months after the transplant.

The invention also provides the use of melflufen, or a salt thereof, for the manufacture of a medicament for the treatment of multiple myeloma in a patient having multiple myeloma who
has not received a stem cell transplant; or
has received a stem cell transplant that was at least 5 years ago; or
is 75 years old or older; or
has not received a stem cell transplant and is 75 years old or older, or has received a stem cell transplant that was at least 5 years ago and is 75 years old or older; or
has received a stem cell transplant and the disease subsequently progressed at least 36 months after the transplant.

The invention also provides the use of melflufen, or a salt thereof, for the manufacture of a medicament for the treatment and/or prophylaxis of multiple myeloma in a patient having multiple myeloma who has not received a stem cell transplant, or in a patient having multiple myeloma who has received a stem cell transplant that was at least 5 years ago. The invention also provides the use of melflufen, or a salt thereof, for the manufacture of a medicament for the treatment and/or prophylaxis of multiple myeloma in a patient having multiple myeloma who is 75 years old or older (and, optionally, has not received a stem cell transplant, or has received a stem cell transplant that was at least 5 years ago).

The invention also provides a kit comprising melflufen and one or more further therapeutic agent(s) (for example selected from dexamethasone, Bortezomib and Daratumumab; preferably dexamethasone) for use in the treatment and/or prophylaxis of multiple myeloma in a patient having multiple myeloma who
has not received a stem cell transplant; or
has received a stem cell transplant that was at least 5 years ago; or
is 75 years old or older; or
has not received a stem cell transplant and is 75 years old or older, or has received a stem cell transplant that was at least 5 years ago and is 75 years old or older; or
has received a stem cell transplant and the disease subsequently progressed at least 36 months after the transplant.

The present invention provides melflufen, or a salt(s) thereof, and dexamethasone for use in the treatment and/or prophylaxis of multiple myeloma in a patient having multiple myeloma who
has not received a stem cell transplant; or
has received a stem cell transplant that was at least 5 years ago; or
is 75 years old or older; or
has not received a stem cell transplant and is 75 years old or older, or has not received a stem cell transplant for at least 5 years and is 75 years old or older; or
has received a stem cell transplant and the disease subsequently progressed at least 36 months after the transplant.

The present invention also provides a method for the treatment or prophylaxis of multiple myeloma, comprising the step of administering melflufen, or a salt thereof, and dexamethasone to a patient having multiple myeloma who has not received a stem cell transplant, or has received a stem cell transplant that was at least 5 years ago, and/or is 75 years old or older, and/or has received a stem cell transplant and the disease subsequently progressed at least 36 months after the transplant. The present invention also provides a pharmaceutical formulation comprising melflufen, or a salt thereof, and dexamethasone for use in the treatment and/or prophylaxis of multiple myeloma in a patient having multiple myeloma who has not received a stem cell transplant, or has received a stem cell transplant that was at least 5 years ago, and/or is 75 years old or older, and/or has received a stem cell transplant and the disease subsequently progressed at least 36 months after the transplant. The invention also provides the use of melflufen, or a salt thereof, and dexamethasone for the manufacture of a medicament for the treatment and/or prophylaxis of multiple myeloma in a patient having multiple myeloma who has not received a stem cell transplant, or has received a stem cell transplant that was at least 5 years ago, and/or is 75 years old or older, and/or has received a stem cell transplant and the disease subsequently progressed at least 36 months after the transplant.

The invention is particularly applicable in a patient having multiple myeloma who
has not received a stem cell transplant; or
has received a stem cell transplant that was at least 5 years ago; or
is 75 years old or older; or
has not received a stem cell transplant and is 75 years old or older, or has not received a stem cell transplant for at least 5 years and is 75 years old or older.

In a particular embodiment, a patient is one having multiple myeloma who has not received a stem cell transplant.

### BRIEF DESCRIPTION OF THE DRAWINGS

FIG. 1 shows a Forest Plot of progression free survival (PFS) unstratified hazard ratios by demographics subgroups for patients in Example 1 that had not had a stem cell transplant and were treated with melflufen+dexamethasone (n=121) or pomalidomide+dexamethasone (n=129).

FIG. 2 shows a Forest Plot of PFS unstratified hazard ratios by disease characteristics subgroups for patients in Example 1 that had not had a stem cell transplant and were treated with melflufen+dexamethasone (n=121) or pomalidomide+dexamethasone (n=129) (BSA median=1.855 m²). The last line of PFS numbers in FIG. 1 also relates to a disease characteristic.

FIG. 3 shows a Forest Plot of Overall Survival (OS) unstratified hazard ratios by demographics subgroups for patients in Example 1 that had not had a stem cell transplant and were treated with melflufen+dexamethasone (n=121) or pomalidomide+dexamethasone (n=129).

FIG. 4 shows a Forest Plot of OS unstratified hazard ratios by disease characteristics subgroups for patients in Example 1 that had not had a stem cell transplant and were treated with melflufen+dexamethasone (n=121) or pomalidomide+dexamethasone (n=129) (BSA median=1.855 m²). The last line of OS numbers in FIG. 3 also relates to a disease characteristic.

FIG. 5 shows a graph of PFS (%) over time for patients in Example 1 that had not had a stem cell transplant and were treated with melflufen+dexamethasone (n=121) or pomalidomide+dexamethasone (n=129), or who had received a stem cell transplant and were treated with melflufen+dexamethasone (n=125) or pomalidomide+dexamethasone (n=120) (^{a} indicates unstratified HR; ^{b} indicates Log-rank P value).

FIG. 6 shows a graph of OS (%) over time for patients in Example 1 that had not had a stem cell transplant and were treated with melflufen+dexamethasone (n=121) or pomalidomide+dexamethasone (n=129) (^{a} indicates unstratified HR; ^{b} indicates Log-rank P value).

FIG. 7 shows a table of PFS hazard ratios and events for patients in Example 1 that had not had a stem cell transplant and were treated with melflufen+dexamethasone (n=121) or pomalidomide+dexamethasone (n=129). It also shows the PFS hazard ratios and events for patients in Example 1 who were treated with melflufen+dexamethasone and had received a stem cell transplant less than 2.5 years ago (n=43), 2.5 to 5 years ago (n=48), or more than 5 years ago (n=34), or who were treated with pomalidomide+dexamethasone and had received a stem cell transplant less than 2.5 years ago (n=35), 2.5 to 5 years ago (n=51), or more than 5 years ago (n=34), (^{a} indicates unstratified HR; ^{b} indicates Log-rank P value).

FIG. 8 shows a table of OS hazard ratios and events for patients in Example 1 that had not had a stem cell transplant and were treated with melflufen+dexamethasone (n=121) or pomalidomide+dexamethasone (n=129). It also shows the OS hazard ratios and events for patients in Example 1 who were, or who were treated with melflufen+dexamethasone and had received a stem cell transplant less than 2.5 years ago (n=43), 2.5 to 5 years ago (n=48), or more than 5 years ago (n=34), or who were treated with pomalidomide+dexamethasone less than 2.5 years ago (n=35), 2.5 to 5 years ago (n=51), or more than 5 years ago (n=34), (^{a} indicates unstratified HR; ^{b} indicates Log-rank P value).

FIG. 9 shows a Forest Plot of Overall Survival (OS) unstratified hazard ratios by demographics and disease characteristics for patients in Example 1 who were treated with melflufen+dexamethasone (n=145) or pomalidomide+dexamethasone (n=148) when patients who had had a stem cell transplant but suffered progression in under 36 months are excluded.

FIG. 10 shows a graph of OS (%) over time for patients in Example 1 who were treated with melflufen+dexamethasone (n=145) or pomalidomide+dexamethasone (n=148) when patients who had had a stem cell transplant but suffered progression in under 36 months are excluded (HR is hazard ratio; ^{a} indicates unstratified HR; ^{b} indicates Log-rank P value).

### DETAILED DESCRIPTION

The inventors have found that melflufen, and in particular melflufen in combination with dexamethasone, is surprisingly effective for the treatment or prophylaxis of multiple myeloma in a patient having multiple myeloma who has not received a stem cell transplant, or in a patient having multiple myeloma who has received a stem cell transplant that was at least 5 years ago. The inventors have further found that melflufen, and in particular melflufen in combination with dexamethasone, is surprisingly effective for the treatment or prophylaxis of multiple myeloma in patients who are 75 years old or older, and, in particular patients who are 75 years old or older and have not received a stem cell transplant, or are 75 years old or older and have received a stem cell transplant that was at least 5 years ago. The inventors have also found that melflufen, and in particular melflufen in combination with dexamethasone, is surprisingly effective for the treatment or prophylaxis of multiple myeloma in a patient who has received a stem cell transplant and the disease subsequently progressed at least 36 months after the transplant.

More particularly, the present inventors have found that melflufen demonstrates superior anti-neoplastic activity in comparison to other treatments for multiple myeloma in these patient populations, and in particular in comparison to treatment with pomalidomide for multiple myeloma in these patient populations. For example, the inventors carried out a randomized, controlled, open-label, Phase 3 multicenter study which enrolled patients with relapsed refectory multiple myeloma following 2-4 lines of prior therapy and who were refractory to lenalidomide in the last line of therapy as demonstrated by disease progression on or within 60 days of completion of the last dose of lenalidomide, wherein patients received either melflufen and dexamethasone, or pomalidomide and dexamethasone as described in Example 1 below. In this study the inventors surprisingly found that patients with multiple myeloma in the trial who had not received a stem cell transplant as a past treatment and who received melflufen had a median progression free survival (PFS) of 9.3 months, whereas patients with multiple myeloma who had not received a stem cell transplant as a past treatment and who received pomalidomide had a median PFS of 4.6 months. Additionally, the inventors found that patients with multiple myeloma in the trial who had not received a stem cell transplant as a past treatment and who received melflufen had a median overall survival (OS) of 21.6 months, whereas patients with multiple myeloma who had not received a stem cell transplant as a past treatment and who received pomalidomide had a median OS of 16.5 months.

This significant improvement was present across demographic and disease subgroups. For example, it was present for patients under 65 years (PFS for the melflufen group: 10.8; PFS for the pomalidomide group: 4.7; OS for the melflufen group: 27.5 OS for the pomalidomide group: NA), those of 64 to 74 years (PFS for the melflufen group: 9.3; PFS for the pomalidomide group: 3.9; OS for the melflufen group: 22.2; OS for the pomalidomide group:

15.0), those of age under 75 years (PFS for the melflufen group: 9.4; PFS for the pomalidomide group: 4.3; OS for the melflufen group: 22.2; OS for the pomalidomide group: 18.2) and those of 75 years and over (PFS for the melflufen group: 9.3; PFS for the pomalidomide group: 4.9; OS for the melflufen group: 14.8; OS for the pomalidomide group: 8.1).

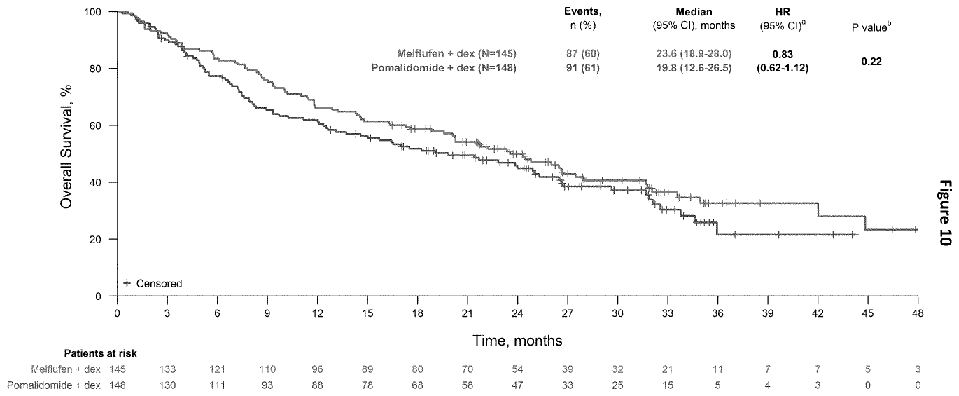

Furthermore, it is also seen in Example 1 that patients who had not had a stem cell transplant as a past treatment or who had had a stem cell transplant as a past treatment and had subsequently suffered progression of disease only 36 or more months after the transplant had a better response rate to melflufen (and dexamethasone) in comparison to other treatments for multiple myeloma in these patient populations, and in particular in comparison to treatment with pomalidomide (and dexamethasone). The inventors found that patients with multiple myeloma in the trial excluding those who had received a stem cell transplant as a past treatment and progressed in under 36 months had a median overall survival (OS) of 23.6 months when treated with melflufen, whereas patients who received pomalidomide had a median OS of 19.8 months. The same finding is also seen in FIG. 10.

The use of melflufen for treating multiple myeloma in patients having multiple myeloma who have not had a stem cell transplant is especially surprising because, to the inventors' knowledge, there are no known multiple myeloma treatments that show especially beneficial effects in this patient population. Similarly, to the inventors' knowledge, there are no known multiple myeloma treatments that show especially beneficial effects in patients who have received a stem cell transplant and the disease subsequently progressed at least 36 months after the transplant. As described above, there remains an intense need for further treatment options in multiple myeloma patients who are not able to have a stem cell transplant. The results reported in Example 1 of the present application show an extremely significant improvement in median PFS and median OS for the multiple myeloma patient population that have not received a stem cell transplant. As multiple myeloma remains incurable and fatal, these surprising results offer patients in this patient population a new treatment that can give them valuable months of time compared to alternative treatment choices.

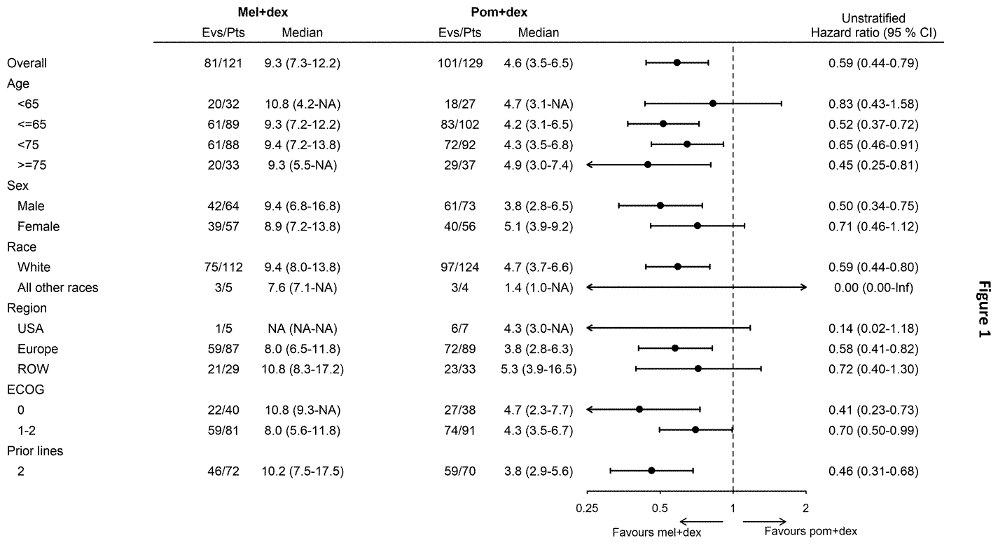

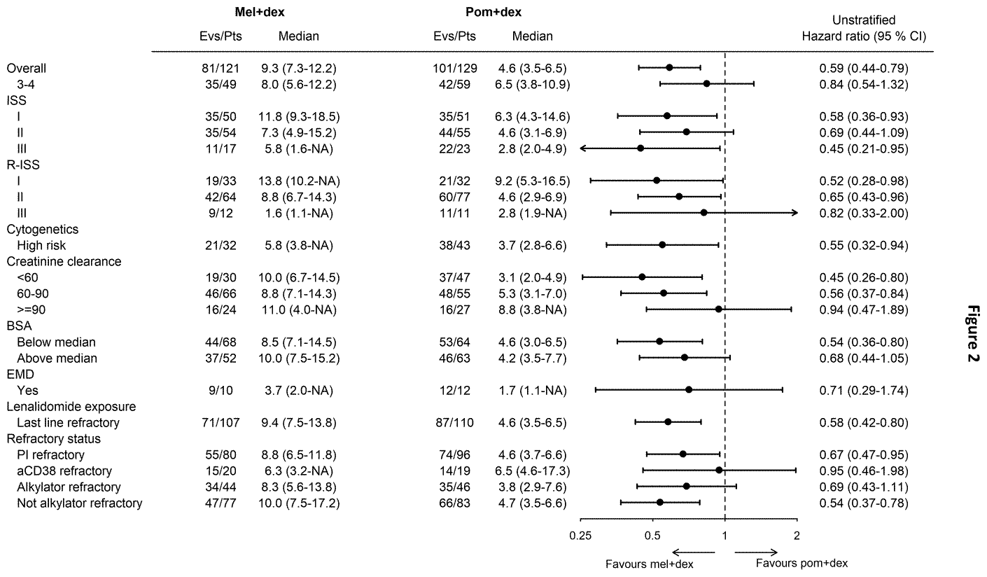

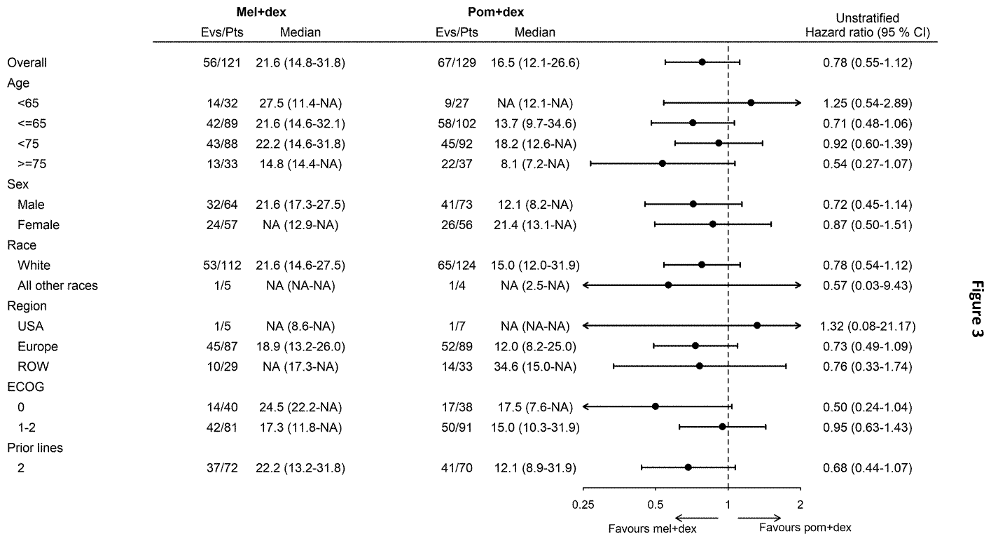

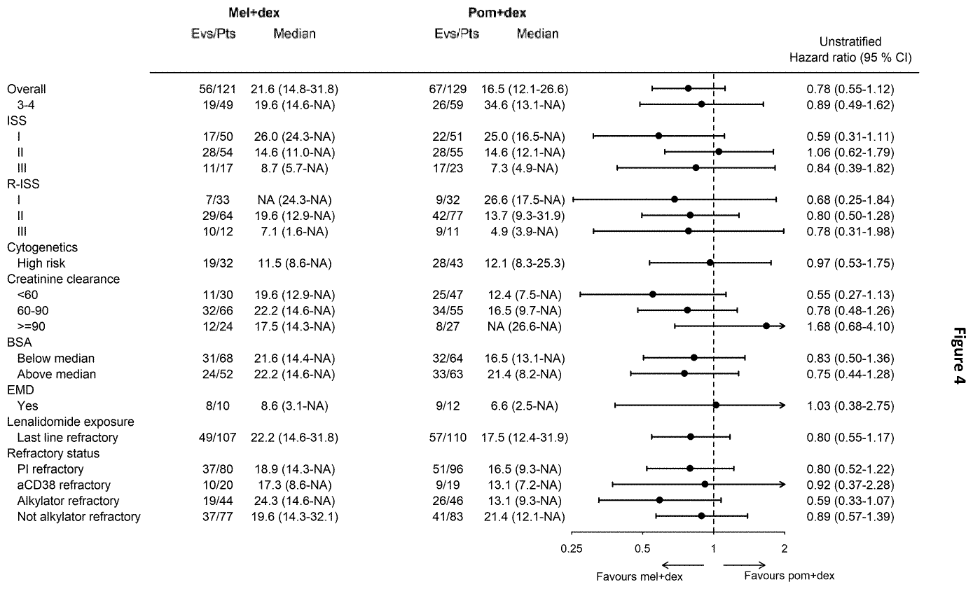

Furthermore, as there are multiple reasons why a patient may not have received a stem cell transplant, it is even more surprising that there are consistent beneficial effects in this patient population and across multiple demographic and disease subgroups within this patient population. As can be seen from FIGS. 1 to 4, which show the PFS and OS unstratified hazard ratios by demographics subgroups (FIGS. 1 and 3, respectively) and disease characteristics subgroups (FIGS. 2 and 4, respectively), the efficacy of the treatment of the present is persistent across multiple myeloma populations who have not had a stem cell transplant.

In the Example 1 results, the hazard ratio is a measure of the relative risk of an event at each time point during follow-up when receiving melflufen in relation to pomalidomide. A value below 1 indicates a better treatment effect for melflufen, and a value above 1 indicates a better treatment effect for pomalidomide. In the Example 1 study the inventors also surprisingly found that patients with multiple myeloma in the trial have received a stem cell transplant that was at least 5 years ago, had a PFS hazard ratio of 0.76 in favour of melflufen compared to pomalidomide treatment, and an OS hazard ratio of 0.87 in favour of melflufen compared to pomalidomide treatment. Additionally, patients with multiple myeloma in the trial who received a stem cell transplant that was 2.5 years to 5 years ago, had a hazard ratio of 0.83 in favour of melflufen compared to pomalidomide treatment. Therefore, these results show that melflufen is especially beneficial for treating patients having multiple myeloma who have received a stem cell transplant that was at least 2.5 years ago, and especially at least 5 years ago. It is also seen that melflufen is especially beneficial for treating patients having multiple myeloma who have received a stem cell transplant and who then did not have disease progression until at least 36 months after the transplant.

The use of melflufen for treating multiple myeloma in patients having multiple myeloma who have received a stem cell transplant that was at least 2.5 years ago, and especially at least 5 years ago, is especially surprising because, to the inventors' knowledge, there are no known multiple myeloma treatments that show especially beneficial effects in these patient populations. As described above, there remains an intense need for further treatment options in multiple myeloma patients who previously received a stem cell transplant, but are not able to have a further stem cell transplant, and these results offer patients in this patient population a new treatment that can give them valuable months of time compared to alternative treatment choices. Similarly, for patients who have received a stem cell transplant and the disease subsequently progressed at least 36 months after the transplant, these results offer patients in this patient population a new treatment that can give them valuable months of time compared to alternative treatment choices.

Finally, the present inventors surprising found that melflufen treatment in terms of both PFS and OS was especially beneficial effects in patients of 75 years old or older.

For example, patients with multiple myeloma in the trial who were 75 or over and received melflufen had a median PFS of 9.4 months, whereas patients with multiple myeloma who had not received a stem cell transplant as a past treatment and who received pomalidomide had a median PFS of 4.6 months. Additionally, the inventors found that patients with multiple myeloma in the trial who were 75 or over and received melflufen had a median overall survival (OS) of 21.6 months, whereas patients with multiple myeloma who had not received a stem cell transplant as a past treatment and who received pomalidomide had a median OS of 8.3 months.

### Melflufen and Salts Thereof

Melflufen (also known as melphalan flufenamide and L-melphalanyl-4-fluoro-L-phenylalanine ethyl ester), is an anti-tumour agent useful in the treatment cancer, particularly the treatment of multiple myeloma. Melflufen, and salts thereof, especially the hydrochloride salt thereof, are known from, for example, WO 01/96367 and WO 2014/065751 (the contents of which are incorporated herein by reference). The structure of the hydrochloride salt of melflufen is shown below:

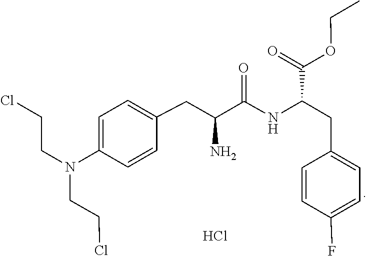 [SMILES: CCOC(=O)[C@H](Cc1ccc(F)cc1)NC(=O)[C@@H](N)Cc1ccc(N(CCCl)CCCl)cc1 ; Cl]

For the avoidance of doubt, in this document, when the term “melflufen” is used, it includes salts of melflufen, as well as isotopic derivatives of melflufen, unless stated otherwise. Isotopic derivatives of melflufen are described in WO 2020/079165, the content of which is incorporated herein by reference.

Also for the avoidance of doubt, when referred to in this document, the mass of melflufen is the mass of the melflufen molecule excluding the mass of any counterion unless explicitly stated otherwise.

Salts of melflufen which are suitable for use in the present invention are those wherein a counterion is pharmaceutically acceptable. Suitable salts include those formed with organic or inorganic acids. In particular, suitable salts formed with acids according to the invention include those formed with mineral acids, strong organic carboxylic acids, such as alkanecarboxylic acids of 1 to 4 carbon atoms which are unsubstituted or substituted, for example, by halogen, such as saturated or unsaturated dicarboxylic acids, such as hydroxycarboxylic acids, such as amino acids, or with organic sulfonic acids, such as (C₁-C₄) alkyl or aryl sulfonic acids which are unsubstituted or substituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxalic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine.

Preferred salts of melflufen include acid addition salts such as those formed from hydrochloric, hydrobromic, acetic, p-toluenesulfonic, tartaric, sulphuric, succinic, phosphoric, oxalic, nitric, methanesulfonic, malic, maleic and citric acid. More preferably, the salt of melflufen for use according to the present invention is the hydrochloride salt (i.e. the addition salt formed from hydrochloric acid).

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. The complex may incorporate a solvent in stoichiometric or non-stoichiometric amounts. Solvates are described in Water-Insoluble Drug Formulation, 2^{nd} ed R. Lui CRC Press, page 553 and Byrn et al Pharm Res 12(7), 1995, 945-954. Before it is made up in solution, the melflufen, or a salt thereof, for use in the present invention may be in the form of a solvate. Solvates of melflufen that are suitable for use according to the present invention are those wherein the associated solvent is pharmaceutically acceptable. For example a hydrate is a pharmaceutically acceptable solvate.

### Formulations

While it is possible for melflufen, and salts thereof, to be administered alone, it is preferable for it to be present in a formulation and particularly in a pharmaceutical formulation. Pharmaceutical formulations include those suitable for oral, parenteral (including subcutaneous, intradermal, intraosseous infusion, intramuscular, intravascular (bolus or infusion), and intramedullary), intraperitoneal, transmucosal, transdermal, rectal and topical (including dermal, buccal, sublingual and intraocular) administration although the most suitable route may depend upon, for example, the condition and disorder of the subject under treatment.

In one embodiment of the invention, melflufen is administered as a pharmaceutical formulation suitable for oral or parenteral (including subcutaneous, intradermal, intraosseous infusion, intramuscular, intravascular (bolus or infusion), and intramedullary) administration.

Pharmaceutical formulations of melflufen suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The melflufen may also be presented as a bolus, electuary or paste. Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:25, 1988.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Preferably the formulations may be presented in unit dosage or divided dosage containers, for example sealed ampoules and vials. The formulation may be stored in a freeze-dried (lyophilised) condition requiring only the addition of the sterile liquid carrier, for example saline, a physiologically acceptable solution or water-for-injection, immediately prior to use. The formulation may also be stored as a liquid pharmaceutical formulation requiring only the addition of the sterile liquid carrier, for example saline, a physiologically acceptable solution or water-for-injection, immediately prior to use.

Extemporaneous injection and infusion solutions and suspensions may be prepared from sterile powders, granules or other dry composition. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono-or diglycerides, and fatty acids, including oleic acid, or Cremaphor.

In a preferred embodiment of the invention, the melflufen for use in the present invention comprises a lyophilized pharmaceutical preparation of a melflufen or a salt thereof. The term “lyophilized pharmaceutical preparation of a melflufen or a salt thereof” is understood to mean that the melflufen or a salt thereof is freeze-dried (“lyophilization”, “lyophilized” etc. may in the present context be used interchangeably with “freeze-drying”, “freeze-dried” etc.). A lyophilized pharmaceutical preparation of melflufen or a salt thereof as described herein may be a white, fluffy powder in contrast to a non-lyophilized melflufen or a pharmaceutically acceptable salt thereof, which is typically in the form of a dense, slightly yellowish powder.

A lyophilized pharmaceutical preparation of melflufen, or a salt thereof, for use in the present invention may comprise sucrose. The inclusion of sucrose provides a lyophilized preparation that is stable as such, and water-soluble, without the presence of an organic solvent, at a sufficient rate compared to the degradation rate, and is thereby useful in therapy and does not have toxicity brought about by the organic solvent. Due to the increased solubility and/or rate of dissolution of melflufen, or a salt thereof, after lyophilization in the presence of sucrose, it is possible to prepare a dissolved melflufen, or a salt thereof, solution, such as a pharmaceutical composition comprising melflufen, or a salt thereof, which has a usefully high concentration of melflufen and which is substantially free from organic solvents. Preparation of a lyophilized pharmaceutical preparation, a lyophilized pharmaceutical composition, and a kit for making such compositions, of melflufen or a salt thereof, is described in detail in WO 2012/146625 and WO 2014/065751, the contents of which are incorporated herein by reference.

A pharmaceutical formulation of melflufen, or a salt thereof, for use in the present invention may comprise a lyophilized pharmaceutical preparation comprising melflufen, or a salt thereof. Preferably, the formulation comprises sucrose. More preferably, the formulation comprises sucrose with a weight ratio (w/w) between melflufen and sucrose of about 1:25 to 1:75, for example 1:50.

Where the formulation is a pharmaceutical solution, it may be prepared from a lyophilized pharmaceutical preparation comprising melflufen, or a salt thereof, and further comprise a physiologically acceptable solvent(s), such as a glucose solution and/or a saline solution.

In another preferred embodiment of the invention, the melflufen for use in the present invention comprises a liquid pharmaceutical preparation of melflufen or a salt thereof. Preferably, the liquid pharmaceutical preparation or consisting essentially of the following components: i) melflufen, or a salt thereof; ii) propylene glycol; iii) optionally one or more physiologically acceptable aqueous solvent(s); and iv) optionally one or more additional therapeutic agent(s); or the the liquid pharmaceutical preparation or consisting essentially of the following components: i) melflufen, or a salt thereof; ii) polyethylene glycol; iii) optionally one or more physiologically acceptable aqueous solvent(s); and iv) optionally one or more additional therapeutic agent(s). Preparation of liquid pharmaceutical formulations, liquid pharmaceutical formulations, and a kit for making such liquid pharmaceutical formulations of melflufen, or a salt thereof, are described in detail in WO 2020/212594, the contents of which are incorporated herein by reference.

It should be understood that in addition to the ingredients particularly mentioned above, the formulations for use in this invention may include other agents conventional in the art having regard to the type of formulation in question.

### Dosage Regimens

Melflufen, or a salt(s) thereof, and pharmaceutical formulations comprising melflufen, find use in the treatment and/or prophylaxis of multiple myeloma in a patient having multiple myeloma who has not received a stem cell transplant.

Melflufen, or a salt(s) thereof, and pharmaceutical formulations comprising melflufen, also find use in the treatment and/or prophylaxis of multiple myeloma in a patient having multiple myeloma who has received a stem cell transplant that was at least 5 years ago. Melflufen, or a salt(s) thereof, and pharmaceutical formulations comprising melflufen, also find use in the treatment and/or prophylaxis of multiple myeloma in a patient having multiple myeloma who is 75 years old or older, and, optionally, has not received a stem cell transplant or has received a stem cell transplant that was at least 5 years ago.

Melflufen, or a salt(s) thereof, and pharmaceutical formulations comprising melflufen, also find use in the treatment and/or prophylaxis of multiple myeloma in a patient having multiple myeloma who has received a stem cell transplant and the disease subsequently progressed at least 36 months after the transplant.

For the avoidance of doubt, when a patient is defined herein as a patient having multiple myeloma who ‘has not received a stem cell transplant, or has received a stem cell transplant that was at least 5 years ago, and/or is 75 years old or older’, or similar wording, that patient may: (i) have not received a stem cell transplant, or (ii) have received a stem cell transplant that was at least 5 years ago, or (iii) be 75 years old or older, or (iv) have not received a stem cell transplant and be 75 years old or older, or (v) have received a stem cell transplant that was at least 5 years ago and be 75 years old or older.

For the avoidance of doubt, when a patient is defined herein as a patient having multiple myeloma who ‘has received a stem cell transplant and the disease subsequently progressed at least 36 months after the transplant’, or similar wording, that patient may also fall in one or more of the groups that (i) have received a stem cell transplant that was at least 5 years ago, or (ii) be 75 years old or older, or (iii) have received a stem cell transplant that was at least 5 years ago and be 75 years old or older.

The amount of melflufen which is required to achieve a therapeutic effect will vary with particular route of administration and the characteristics of the subject under treatment, for example the species, age, weight, sex, medical conditions, the particular disease and its severity, and other relevant medical and physical factors. An ordinarily skilled physician can readily determine and administer the effective amount of melflufen required for treatment or prophylaxis of multiple myeloma in a patient having multiple myeloma who has not received a stem cell transplant, or has received a stem cell transplant that was at least 5 years ago, and/or is 75 years old or older.

Melflufen may be administered daily, every second or third day, weekly, every second, third or fourth week or even as a high single dose depending on the subject and severity of the multiple myeloma to be treated.

In one embodiment of the invention, the melflufen, or salt thereof, may be administered in an amount of about 1 to 150 mg (excluding the mass of any counterion). For example, 1, 5, 10, 15, 20, 25, 40, 45, 50, 60, 70, 80, 90, 100, 110, 120, 130, 140 or 150 mg (excluding the mass of any counterion). Preferably, the dosage of melflufen or salt thereof, per administration is 1 to 50 mg (excluding the mass of any counterion), for example 1, 5, 10, 15, 20, 25, 30, 35, 40, 45 or 50 mg. More preferably, the dosage of melflufen or salt thereof, per administration is 1 to 40 mg (excluding the mass of any counterion), for example 1, 5, 10, 15, 20, 25, 30, 35, or 40 mg. For example, the dosage of melflufen or salt thereof, per administration is 10 to 40 mg (excluding the mass of any counterion), for example 10, 15, 20, 25, 30, 35, or 40 mg . In certain embodiments, the dosage of melflufen or salt thereof, per administration may be 10 to 20 mg, 20 to 30 mg, or 30 to 40 mg.

In one embodiment of the invention, the melflufen, or salt thereof, may be administered in an amount of about 35.0 to 45.0 mg of melflufen, preferably 36.0 to 44.0 mg, preferably 37.0 to 43.0 mg, preferably 37.5 to 42.5 mg (for example 37.5, 38.0, 38.5, 39.0, 39.5, 40.0, 40.5, 41.0, 41.5, 42.0 or 42.5 mg), more preferably 38.0 to 42.0 mg; and most preferably 39.0 to 41.0 mg (for example 39.0, 39.5, 40.0, 40.5 or 41.0 mg more preferably 39.5, 40.0 or 40.5 mg and most preferably 40.0 mg). The melflufen, or salt thereof, may be administered, for example, as a parenteral dosage over 25-35 minutes.

In embodiments where melflufen is in the form of its hydrochloride (HCI) salt, an amount of about 37.6 to 48.3 mg, preferably 39.0 to 47.0 mg, more preferably 41.0 to 45.0 mg, more preferably 42.5 to 43.5 mg and most preferably 42.9 mg, of melflufen hydrochloride (including the mass of the salt component), is administered. The melflufen hydrochloride may be administered, for example, as a parenteral dosage over 25-35 minutes.

Preferably the melflufen, or salt thereof, of the present invention is administered over 26 to 34 minutes, more preferably over 27 to 33 minutes, even more preferably over 28 to 32 minutes, even more preferably over 29 to 31 minutes, and most preferably over 30 minutes.

Melflufen, or a salt thereof, may be administered as a parenteral or oral dosage. In a preferred embodiment of the invention, melflufen or a salt thereof, is administered as a parenteral dosage. As such, pharmaceutical formulations useful according to the invention are those suitable for parenteral administration.

Parenteral administration includes intravenous (into a vein, for example a central or a peripheral vein) (bolus or infusion), intra-arterial (into an artery, for example a central or a peripheral artery), intraosseous infusion (into the bone marrow), intra-muscular (into muscle), intradermal (into the dermis), and subcutaneous (under the skin) administration. Preferably, the dosage of the present invention is administered intravenously or intra- arterially, and more preferably by intravenous infusion (for example central intravenous infusion or peripheral intravenous infusion). As such, pharmaceutical formulations especially useful for the present invention are those suitable for intravenous administration, and more especially intravenous infusion.

In one embodiment of the invention, the dosage of melflufen (excluding the mass of any counterion) is administered as a parenteral dosage at an infusion rate of around 0.3 to 1.8 mg/min, for example 0.5 to 1.8 mg/min, for example 0.8 to 1.8 mg/min, for example 1.0 to 1.8 mg/min, for example 1.1 to 1.8 mg/min, for example 1.1 to 1.7 mg/min, for example 1.1 to 1.6 mg/min, for example 1.2 to 1.6 mg/min, or for example 1.2 to 1.5 mg/min. In one embodiment, the dosage of melflufen (excluding the mass of any counterion) is administered as a parenteral dosage at an infusion rate of around 1.2 to 1.4 mg/min (for example 1.2, 1.3 or 1.4 mg/min).

In an embodiment of the invention, a dosage of melflufen of around 1 to 50 mg (excluding the mass of the counterion) (for example 1 to 45 mg) is administered as a parenteral dosage over around 5-35 minutes. For example, a dosage of melflufen of 40 mg (excluding the mass of the counterion) and as a parenteral dosage over around 30 minutes, or for example, a dosage of melflufen of 20 mg (excluding the mass of the counterion) and as a parenteral dosage over around 15 minutes, or for example, a dosage of melflufen of 10 mg (excluding the mass of the counterion) and as a parenteral dosage over around 7.5 minutes.

As regards the dosage of melflufen for use in the present invention, when a mass of melflufen or a salt thereof is referred to, that is the mass when no counterion is included in the calculation of the dosage mass of the melflufen. The molecular weight of counterion-free melflufen is 498.42 g/mol. For a dosage of a salt of melflufen, the actual dosage mass administered to the patient must take into account the mass of the counterion. This is routine for the person skilled in the art.

For example, when the melflufen is in the form of its hydrochloride (HCl) salt (which has a molecular weight of 534.88 g/mol), the equivalent dosage rate to 1.1 to 1.8 mg/min for melflufen hydrochloride (including the mass of the counterion) will be 1.2 to 1.9 mg/min. For a dosage of melflufen of 1 to 50 mg, the equivalent dosage of melflufen hydrochloride will be approximately 1.1 to 53.8 mg. For a dosage of melflufen of 10 to 45 mg, the equivalent dosage of melflufen hydrochloride will be approximately 10.7 to 48.3 mg.

When melflufen, or a salt thereof, is administered as a parenteral dosage, the dosage of melflufen must be in the form of a liquid, for example a solution or suspension comprising the melflufen.

Preferably the melflufen, or salt thereof, of the present invention is taken as part of a treatment cycle. In a cycle, the melflufen may be administered on day 1 of the cycle, wherein the cycle lasts X days, with no further melflufen administered for the next X-1 days. X may be, for example, from 1 to 42, from 5 to 42, from 7 to 42, from 10 to 42, or from 14 to 42. Preferably X may be from 14 to 35 days, more preferably from 21 to 35 days, and more preferably 21 to 30 days; for example 21 days, 28 days, 29 days, 30 days or 35 days. In certain embodiments X may be, for example, 21 to 30 days, 21 to 28 days, 28 to 30 days, or 28 to 29 days. For the avoidance of doubt, a dose of melflufen administered in a treatment cycle may be a dose as described elsewhere in the present application, for example a dose of 1 to 50 mg melflufen.

In a preferred embodiment of the invention, melflufen, or a salt thereof, is administered on day 1 of a 21 day cycle followed by 20 days of rest with no further melflufen being administered during that time; or administered on day 1 of a 28 day cycle followed by 27 days of rest with no further melflufen being administered during that time; or administered on day 1 of a 29 day cycle followed by 28 days of rest with no further melflufen being administered during that time; or administered on day 1 of a 30 day cycle followed by 29 days of rest with no further melflufen being administered during that time; or administered on day 1 of a 35 day cycle followed by 34 days of rest with no further melflufen being administered during that time. In certain embodiments, the treatment cycle is 28 days; or administered on day 1 of a 42 day cycle followed by 41 days of rest with no further melflufen being administered during that time. In certain embodiments, the treatment cycle is 28 days.

The cycle may be repeated one or several times depending on the category (for example high grade or low grade), class (for example relapsed, refractory, etc.) or stage of the multiple myeloma. For example, the cycle may be repeated from 1 to 15 times, for example from 2 to 12 times, for example 2 to 7 times, for example 2, 3, 4, 5, 6 or times. The cycle may be repeated, 3, 4 or 5 times.

An ordinarily skilled physician or clinician can readily determine the number of cycles of melflufen, or a salt thereof, required to treat, prevent, counter or arrest the progress of the multiple myeloma in a patient having multiple myeloma who has not received a stem cell transplant, or has received a stem cell transplant that was at least 5 years ago, and/or is 75 years old or older. An ordinarily skilled physician or clinician can readily determine the number of cycles of melflufen, or a salt thereof, required to treat, prevent, counter or arrest the progress of the multiple myeloma in a patient having multiple myeloma who has received a stem cell transplant and the disease subsequently progressed at least 36 months after the transplant.

The dosage regimens of the invention are particularly safe and effective for the treatment and prophylaxis of multiple myeloma in a patient with multiple myeloma, or at risk of developing multiple myeloma, who has not received a stem cell transplant, or has received a stem cell transplant that was at least 5 years ago, and/or is 75 years old or older. Similarly, the dosage regimens of the invention are particularly safe and effective for the treatment and prophylaxis of multiple myeloma in a patient with multiple myeloma, or at risk of developing multiple myeloma, who has received a stem cell transplant and the disease subsequently progressed at least 36 months after the transplant.

Whilst melflufen, or a salt thereof, may be used as the sole active ingredient in the present invention, it is also possible for it to be used in combination with one or more further therapeutic agent(s), and the use of such combinations provides one preferred embodiment of the invention.

Such further therapeutic agents may be agents useful in the treatment or prophylaxis of multiple myeloma, or other pharmaceutically active materials. Such agents are known in the art. Examples of further therapeutic agents for use in the present invention include steroids (prednisone and dexamethasone), IMiDs (thalidomide, lenalidomide and pomalidomide), PIs (bortezomib, carfilzomib and ixazomib), histone deacetylase (HDAC) inhibitors (panobinostat), conventional chemotherapy (alkylators (e.g. melphalan, cyclophosphamide, bendamustine), doxorubicin), anti-CD38 antibodies (daratumumab) and anti-SLAMF7 antibodies (elotuzumab); for example steroids (prednisone and dexamethasone), IMiDs (thalidomide, lenalidomide and pomalidomide), PIs (bortezomib and carfilzomib), histone deacetylase (HDAC) inhibitors (panobinostat) and conventional chemotherapy (alkylators (e.g. melphalan, cyclophosphamide) and doxorubicin). Examples of further therapeutic agents for use in the present invention also include antibodies against the B-cell maturation antigen (belantamab), inhibitors of nuclear export (selinexor) and autologous chimeric antigen receptor (CAR) T-cell therapy directed against the B-cell maturation antigen (ciltacabtagene). Preferred further therapeutic agents for use in the present invention include dexamethasone, pomalidomide and bortezomib. For example, further therapeutic agents for use in the present invention include dexamethasone; or dexamethasone and pomalidomide; or dexamethasone and bortezomib.

Thus, the invention also provides melflufen, or a salt thereof, together with one or more further therapeutic agent(s) for use in the treatment or prophylaxis of multiple myeloma in a patient having multiple myeloma who has not received a stem cell transplant, or has received a stem cell transplant that was at least 5 years ago, and/or is 75 years old or older, wherein a dosage of melflufen is administered at a rate of 1.0 to 1.8 mg/min. For example a dosage of 35 to 45 mg (preferably 37.5 to 42.5 mg, more preferably 39 to 41 mg and most preferably 40 mg) is administered as a parenteral dosage over 25-35 minutes (preferably over 30 minutes). Preferably the further therapeutic agent is dexamethasone. In another embodiment, the one or more further therapeutic agent(s) are selected from the group consisting of dexamethasone, pomalidomide and bortezomib. For example, dexamethasone; or dexamethasone and pomalidomide; or dexamethasone and bortezomib.

The invention further provides melflufen hydrochloride, together with one or more further therapeutic agent(s), for use in the treatment or prophylaxis of multiple myeloma in a patient having multiple myeloma who has not received a stem cell transplant, or has received a stem cell transplant that was at least 5 years ago, and/or is 75 years old or older, wherein a dosage of melflufen hydrochloride (including the mass of the salt) is administered at a rate of 1.1 to 1.9 mg/min. For example as dosage of melflufen hydrochloride (including the mass of the salt) of 37.6 to 48.3 mg (preferably 40 to 45 mg, more preferably 42.9 mg), is administered as a parenteral dosage over 25-35 minutes (preferably over 30 minutes). Preferably the further therapeutic agent is dexamethasone. In another embodiment, the one or more further therapeutic agent(s) are selected from the group consisting of dexamethasone, pomalidomide and bortezomib. For example, dexamethasone; or dexamethasone and pomalidomide; or dexamethasone and bortezomib.

In one very preferred embodiment, the invention provides melflufen, or a salt thereof, and dexamethasone for use in the treatment or prophylaxis of multiple myeloma in a patient having multiple myeloma who has not received a stem cell transplant, has received a stem cell transplant that was at least 5 years ago, and/or is 75 years old or older, wherein a dosage of melflufen is administered at a rate of 1.0 to 1.8 mg/min. For example a dosage of 35 to 45 mg (preferably 37.5 to 42.5 mg, more preferably 39 to 41 mg and most preferably 40 mg) is administered as a parenteral dosage over 25-35 minutes (preferably over 30 minutes). For example, the invention provides melflufen hydrochloride, and dexamethasone, for use in the treatment or prophylaxis of multiple myeloma in a patient having multiple myeloma who has not received a stem cell transplant, wherein a dosage of melflufen hydrochloride (including the mass of the salt) is administered at a rate of 1.1 to 1.9 mg/min. For example as dosage of melflufen hydrochloride (including the mass of the salt) of 37.6 to 48.3 mg (preferably 40 to 45 mg, more preferably 42.9 mg), is administered as a parenteral dosage over 25-35 minutes (preferably over 30 minutes).

When used in a combination, the precise dosage of the other pharmaceutically active material may vary with the dosing schedule, the potency of the particular agent chosen, the age, size, sex and condition of the subject (typically a mammal, for example a human), the nature and severity of the melanoma, and other relevant medical and physical factors.

The above therapeutic agents, when employed in combination with melflufen or a salt thereof, may be used, for example, in those amounts indicated in the Physicians' Desk Reference (PDR) or as otherwise determined by one of ordinary skill in the art.

Where the further therapeutic agent is dexamethasone, preferably the dosage is from 1 mg to 200 mg, preferably 5 mg to 100 mg, more preferably 10 mg to 80 mg, and most preferably 20 mg to 60 mg, for example 40 mg or 20 mg.

In one preferred embodiment, the patient having multiple myeloma has not received a stem cell transplant or has received a stem cell transplant that was at least 5 years ago, and the further therapeutic agent is dexamethasone, and the dosage of dexamethasone is 40 mg or 20 mg. Optionally, the patient may be 75 years old or older.

In one preferred embodiment, the patient having multiple myeloma is 75 years old or older, and, optionally, has not received a stem cell transplant or has received a stem cell transplant that was at least 5 years ago, and the further therapeutic agent is dexamethasone, and the dosage of dexamethasone is 20 mg.

The one or more further therapeutic agent(s) (for example, the dexamethasone) may be used simultaneously, sequentially or separately with/from the administration of the dosage of the melflufen, or salt thereof. The individual components of such combinations can be administered separately at different times during the course of therapy or concurrently in divided or single combination forms.

Where the further therapeutic agent is dexamethasone, preferably the dexamethasone is administered on the same day and simultaneously, sequentially or separately from the administration of the melflufen, or salt thereof. More preferably it is administered separately from and on the same day as the melflufen, or salt thereof.

For example, when the melflufen, or a salt thereof, for use in the present invention is taken as part of a treatment cycle (for example melflufen, or a salt thereof is administered on day 1 of a cycle lasting X days, with no further melflufen taken for the next X-1 days), the dexamethasone may be administered simultaneously, sequentially or separately on the same day as the melflufen is administered (i.e. on day 1). X may be, for example, from 14 to 42, preferably from 14 to 35 days, and more preferably from 21 to 28 days; for example 21 days or 28 days.

In one preferred embodiment of the invention dexamethasone is administered on day 1 in a treatment cycle. More preferably dexamethasone is also administered weekly during such a treatment cycle, for example administered on days 1, 8 and 15 of a 21 day cycle; or on days 1, 8, 15 and 22 of a 28 day cycle.

In another preferred embodiment, melflufen, or a salt thereof is administered, according to the present invention, on day 1 of a 21 day cycle, and dexamethasone is administered simultaneously, sequentially or separately on day 1 of the cycle, followed by 20 days of rest with no further melflufen being administered during that time; or administered, according to the present invention, on day 1 of a 28 day cycle, and dexamethasone is administered simultaneously, sequentially or separately on day 1 on the cycle, followed by 27 days of rest with no further melflufen being administered during that time. Preferably the cycle is 28 days. Preferably, the dexamethasone is administered separately from the melflufen, or salt thereof, on day 1. Preferably the dexamethasone is administered orally or intravenously. Preferably the dose of dexamethasone is 20 mg or 40 mg.

In another preferred embodiment, when melflufen, or a salt thereof, for use in the present invention taken as part of a cycle (e.g. melflufen is administered on day 1 of a cycle lasting X days, with no further melflufen taken for the next X-1 days), the dexamethasone is administered simultaneously, sequentially or separately on the same day as the melflufen is administered (i.e. on day 1), and weekly thereafter during the cycle. For example dexamethasone is administered on day 1, 8, 15, 22, 29 etc. depending on the length of the cycle. X may be, for example, from 14 to 42, preferably from 14 to 35 days, and more preferably from 21 to 28 days; for example 21 days or 28 days.

In such an embodiment, melflufen, or a salt thereof is administered, according to the present invention, on day 1 of a 21 day cycle, followed by 20 days of rest with no further melflufen being administered during that time, and dexamethasone is administered simultaneously, sequentially or separately on day 1 on the cycle and on days 8 and 15 of the 21 day cycle; or melflufen, or a salt thereof is administered, according to the present invention, on day 1 of a 28 day cycle, followed by 27 days of rest with no further melflufen being administered during that time, and dexamethasone is administered simultaneously, sequentially or separately on day 1 on the cycle and on days 8, 15 and 22 of the 28 day cycle. Preferably, the dexamethasone is administered separately to the melflufen, or salt thereof, on day 1 as an oral dosage or an intravenous dosage (preferably an oral dosage). The later dosage of dexamethasone may be oral dosages or intravenous dosages (preferably the later dosages are oral dosages).

It is noted that the preferred aspects of this invention recited in respect of the compound of the invention and its use are equally applicable to the method of treatment of the present invention and the method of manufacture of the present invention.

### Kits

The present invention provides a kit comprising melflufen, or a salt(s) thereof, and one or more further therapeutic agents that are useful in the treatment or prophylaxis of multiple myeloma in a multiple myeloma patient who has not received a stem cell transplant.

The present invention also provides a kit comprising melflufen, or a salt(s) thereof, and one or more further therapeutic agents that are useful in the treatment or prophylaxis of multiple myeloma in a multiple myeloma patient who has received a stem cell transplant that was at least 5 years ago.

The present invention provides a kit comprising melflufen, or a salt(s) thereof, and one or more further therapeutic agents that are useful in the treatment or prophylaxis of multiple myeloma in a multiple myeloma patient who is 75 years old or older, and, optionally, has not received a stem cell transplant or has received a stem cell transplant that was at least 5 years ago.

Examples of further therapeutic agents for use in the present invention include steroids (e.g. prednisone and dexamethasone), IMiDs (e.g. thalidomide, lenalidomide and pomalidomide), PIs (e.g. bortezomib, carfilzomib, and ixazomib), histone deacetylase (HDAC) inhibitors (e.g. panobinostat), conventional chemotherapy (e.g. melphalan, cyclophosphamide, doxorubicin, bendamustine), anti-CD38 antibodies (daratumumab) and anti-SLAMF7 antibodies (elotuzumab). Examples of further therapeutic agents for use in the present invention also include antibodies against the B-cell maturation antigen (belantamab), inhibitors of nuclear export (selinexor) and autologous chimeric antigen receptor (CAR) T-cell therapy directed against the B-cell maturation antigen (ciltacabtagene).

In a preferred embodiment of the invention, at least one of the one or more further therapeutic agent(s) included in the kit of the invention is a steroid (e.g. prednisone and dexamethasone), PI (e.g. bortezomib, carfilzomib, and ixazomib), or anti-CD38 antibody (daratumumab). In a preferred embodiment of the invention, at least one of the one or more further therapeutic agent(s) included in the kit of the invention is dexamethasone, bortezomib, or daratumumab.

In one embodiment the kit may comprise melflufen, or a salt(s) thereof, dexamethasone, and optionally further comprise bortezomib and/or daratumumab. In one embodiment the kit may comprise melflufen, or a salt(s) thereof, and dexamethasone.

The kit of the present invention finds use in the uses and methods of treatment and/or prophylaxis of the present invention, as described herein.

For the avoidance of doubt, the melflufen, or salt thereof, is present in a kit according to the present invention in a form and quantity suitable for use according to the present invention. Suitable pharmaceutical formulations are described herein. The skilled person can readily determine a quantity of the melflufen, or a salt thereof, and the suitable the one or more further therapeutic agent(s) for the use according the present invention.

### Multiple Myeloma

The treatment of the present invention is useful for the treatment of multiple myeloma in a patient who has not received a stem cell transplant. The treatment of the present invention is also useful for the treatment of multiple myeloma in a patient who has received a stem cell transplant that was at least 5 years ago. The treatment of the present invention is also useful for the treatment of multiple myeloma in a patient who is 75 years old or older, and, optionally, has not received a stem cell transplant or has received a stem cell transplant that was at least 5 years ago. The treatment of the present invention is also useful for the treatment of multiple myeloma in a patient who has received a stem cell transplant and the disease subsequently progressed at least 36 months after the transplant.

There are several categories of multiple myeloma, including monoclonal gammopathy of undetermined significance (MGUS), asymptomatic myeloma (further subdivided into smoldering myeloma or indolent myeloma), and symptomatic myeloma. Multiple myeloma may be classed as primary, refractory, relapsed and relapsed-refractory.

Relapsed multiple myeloma (also known as recurrent myeloma) can be defined as multiple myeloma that recurs on or within 60 days of last dosage of treatment. Relapsed MM is generally regarded as a recurrence of the disease after prior response to treatment.

Refractory multiple myeloma can be defined as multiple myeloma that is not responsive to a specific treatment. Refractory myeloma may occur in patients who never see a response from their treatment therapies or it may occur in patients who do initially respond to treatment, but do not respond to the same treatment after relapse.

Relapsed-refractory multiple myeloma (RRMM) is a specific sub-type of refractory multiple myeloma, and can be defined as multiple myeloma that initially responds to treatment, but does not respond to treatment after relapse. For example, RRMM can be defined as multiple myeloma that recurs within 60 days of the last dosage of a treatment and that initially responds to a treatment, but does not respond to the same treatment after relapse. Relapsed-refractory multiple myeloma is occasionally referred to as refractory-relapsed multiple myeloma.

There are currently 7 classes of approved drugs available for the treatment of MM, namely steroids (e.g. prednisone and dexamethasone), IMiDs (e.g. thalidomide, lenalidomide and pomalidomide), PIs (e.g. bortezomib, carfilzomib, and ixazomib), histone deacetylase (HDAC) inhibitors (e.g. panobinostat), conventional chemotherapy (e.g. melphalan, cyclophosphamide, doxorubicin, bendamustine), anti-CD38 antibodies (daratumumab) and anti-SLAMF7 antibodies (elotuzumab). Also approved are antibodies against the B-cell maturation antigen (belantamab), inhibitors of nuclear export (selinexor) and autologous chimeric antigen receptor (CAR) T-cell therapy directed against the B-cell maturation antigen (ciltacabtagene).

Patients presenting with symptomatic active MM receive primary induction therapy. Those under the age of approximately 65 and in otherwise good health are also considered for consolidation therapy with stem cell transplantation (in particular autologous stem cell transplantation) to enhance remission duration (Moreau, P., et al, J Clin Oncol (2011), Vol 29, pages 1898-1906; Rosinol, L., et al, Expert Rev Hematol (2014) Vol 7, pages 43-53.). The type of induction therapy will vary greatly depending on age, disease status and presence of other comorbidities. The NCCN Guidelines for Multiple Myeloma (NCCN (2019). “NCCN Guidelines for Patients.” National Comprehensive Cancer Network; https://www.nccn.org/patients/guidelines/content/PDF/myeloma-patient.pdf) provide a list of regimens recommended as primary therapy for transplant eligible and non-transplant eligible patients. Regimens including bortezomib and lenalidomide are most often used as primary therapy; these agents are often combined with an alkylator for non-transplant candidates. There are several treatment regimens recommended for patients ineligible for standard stem-cell transplantation in the consensus statement by the International Myeloma Working Group 2014 (Palumbo, A., et al, J Clin Oncol (2014) Vol 32, pages 587-600). Invariably, relapse occurs following each of these agents and salvage therapy is needed.

Refractory multiple myeloma (and/or RRMM) may be refractory to at least one drug from a class of drugs selected from protease inhibitors (PIs), immunomodulatory drugs (IMiDs), alkylators, or anti-CD38 antibody. Some refractory multiple myeloma (and/or RRMM) will be refractory to one or more (for example 1, 2, 3, 4 or 5 or more) drug from two of more classes of drugs selected from protease inhibitors (PIs), immunomodulatory drugs (IMiDs), alkylators or anti-CD38 antibodies. Refractory multiple myeloma (and/or RRMM) may even be refractory to two or more drugs from two or more classes of drugs selected from protease inhibitors (PIs), immunomodulatory drugs (IMiDs), alkylators and anti-CD38 antibodies.

The choice of treatment for any individual with disease relapse will depend on a number of variables, including response and duration to initial chemotherapy, comorbidities, marrow reserve and whether the patient experiences an indolent or aggressive relapse. The selection of treatment in RRMM is especially challenging. Multiple therapies and combinations of some of the approved drugs mentioned above are available for the treatment of RRMM. In general, myeloma patients will receive an average of 4 to 8 different regiments during their lifespan. However, despite the availability of effective therapies, the optimal combinations and sequencing of these agents with other therapies and with one another is still unclear. Ultimately patients relapse from all currently available options.

In many cases, the same agents used as induction therapy may be reinstituted for relapsed disease if the disease recurred more than 6 to 12 months after the last therapy ended. However, if the relapse is after shorter duration, the patient is refractory to initial therapy, or the disease is associated with severe symptoms like renal failure or hypercalcemia, a regimen with different mechanism of action (class switch) is often selected.

Melflufen, or a salt thereof, for use according to the present invention is applicable to any of the aforementioned categories and classes of multiple myeloma in a patient who has not received had a stem cell transplant, or has received a stem cell transplant that was at least 5 years ago, and/or is 75 years old or older, or has received a stem cell transplant and the disease subsequently progressed at least 36 months after the transplant. It is especially useful for a patient who has not received a stem cell transplant.

Melflufen, or a salt thereof, for use according to the present invention is very effective in the treatment of refractory, relapsed and relapsed-refractory multiple myeloma in a patient who has not received a stem cell transplant, or has received a stem cell transplant that was at least 5 years ago, and/or is 75 years old or older (and especially a patient who has not received a stem cell transplant).

For example, melflufen, or a salt thereof, for use according to the present invention is useful for a patient who has not received a stem cell transplant, or has received a stem cell transplant that was at least 5 years ago, or has received a stem cell transplant and the disease subsequently progressed at least 36 months after the transplant, and/or is 75 years old or older (and especially a patient who has not received a stem cell transplant), and is refractory (e.g. refractory or relapsed-refractory) to a protease inhibitor (PIs), immunomodulatory drug (IMiDs), alkylator or anti-CD38 antibody.

Melflufen, or a salt thereof, for use according to the present invention is especially useful for a patient who has not received a stem cell transplant, or has received a stem cell transplant that was at least 5 years ago, or has received a stem cell transplant and the disease subsequently progressed at least 36 months after the transplant, and/or is 75 years old or older (and especially a patient who has not received a stem cell transplant), and that is refractory (e.g. refractory or relapsed-refractory) to an alkylator, for example one or more of low dose melphalan, high dose melphalan and cyclophosphamide. It is especially useful for a patient who has not received a stem cell transplant, or has received a stem cell transplant that was at least 5 years ago, or has received a stem cell transplant and the disease subsequently progressed at least 36 months after the transplant, and/or is 75 years old or older (and especially a patient who has not received a stem cell transplant), and that is refractory (e.g. refractory or relapsed-refractory) to an anti-CD38 antibody. It is very especially useful for a patient who has not received a stem cell transplant, or has received a stem cell transplant that was at least 5 years ago, or has received a stem cell transplant and the disease subsequently progressed at least 36 months after the transplant, and/or is 75 years old or older (and especially a patient who has not received a stem cell transplant), and that is refractory (e.g. refractory or relapsed-refractory) to lenalidomide, and in particular refractory (e.g. refractory or relapsed-refractory) to lenalidomide wherein lenalidomide was the last treatment that the patient received for multiple myeloma. It is also useful for a patient who has not received a stem cell transplant, or has received a stem cell transplant that was at least 5 years ago, or has received a stem cell transplant and the disease subsequently progressed at least 36 months after the transplant, and/or is 75 years old or older (and especially a patient who has not received a stem cell transplant), and is refractory to one or more (for example 1, 2, 3, 4 or 5 or more) drug from two of more classes of drugs selected from protease inhibitors (PIs), immunomodulatory drugs (IMiDs), alkylators and anti-CD38 antibodies.

It is also especially useful for a patient who has not received a stem cell transplant, or has received a stem cell transplant that was at least 5 years ago, or has received a stem cell transplant and the disease subsequently progressed at least 36 months after the transplant, and/or is 75 years old or older (and more especially a patient who has not received a stem cell transplant), and is refractory to one or more (for example 1, 2, 3, 4 or 5 or more) drug from two of more classes of drugs selected from protease inhibitors (PIs), immunomodulatory drugs (IMiDs), alkylators and anti-CD38 antibodies.

Melflufen, or a salt thereof, for use according to the present invention is also especially useful in patients that are refractory (e.g. refractory or relapsed-refractory) to at least one immunomodulatory drug (IMiDs), and more especially in patients that are refractory (e.g. refractory or relapsed-refractory) to at least the immunomodulatory drug lenalidomide, and more especially to at least lenalidomide and 1, 2, 3 or 4 other drugs, for example at least one drug selected from protease inhibitor (PI), immunomodulatory drug (IMiD) alkylators and anti-CD38 antibody. For example, 2, 3 or 4 other drugs including at least one protease inhibitor (PI) and immunomodulatory drug (IMiD). For example, the patient with multiple myeloma may have received at least three prior lines of therapies and have disease that is refractory to at least one proteasome inhibitor, one immunomodulatory agent, and one anti-CD38 monoclonal antibody, and who has demonstrated disease progression on or after the last therapy. If the patient has had a prior autologous stem cell transplantation (ASCT), the time to progression should be at least 3 years from that transplantation.

Melflufen, or a salt thereof, for use according to the present invention is also especially useful in patients that have a median body surface area (BSA) of ≤1.855 m².

In one especially preferred embodiment, the present invention is useful for the treatment of multiple myeloma in a patient who has not received a stem cell transplant, has RRMM, is refractory to the immunomodulatory lenalidomide, and who has received 2, 3 or 4 previous lines of therapy (for example 3 previous lines of therapy).

In another embodiment, present invention is useful for the treatment of multiple myeloma in a patient who has received a stem cell transplant that was at least 5 years ago, has RRMM, is refractory to the immunomodulatory lenalidomide, and who has received 2, 3 or 4 previous lines of therapy (for example 3 previous lines of therapy).

In another embodiment, present invention is useful for the treatment of multiple myeloma in a patient who is at least 75 years old or older (and, for example who has received a stem cell transplant that was at least 5 years ago or has not received a stem cell transplant), has RRMM, is refractory to the immunomodulatory lenalidomide, and who has received 2, 3 or 4 previous lines of therapy (for example 3 previous lines of therapy).

In another embodiment, present invention is useful for the treatment of multiple myeloma in a patient who has received a stem cell transplant and the disease subsequently progressed at least 36 months after the transplant, has RRMM, is refractory to the immunomodulatory lenalidomide, and who has received 2, 3 or 4 previous lines of therapy (for example 3 previous lines of therapy).

Three lines of previous therapy may, for example, be one proteasome inhibitor (eg bortezomib, carfilzomib or ixazomib), one immunomodulatory agent (lenalidomide, pomalidomide or thalidomide) and one anti-CD38 monoclonal antibody (eg daratumumab).

Melflufen, or a salt thereof, for use according to present invention is also useful in patients that are refractory (e.g. refractory or relapsed-refractory) to at least pomalidomide and/or daratumumab.

The present invention is especially beneficial for a patient having multiple myeloma who has not received a stem cell transplant, or has received a stem cell transplant that was at least 5 years ago, or has received a stem cell transplant and the disease subsequently progressed at least 36 months after the transplant (and especially a patient who has not received a stem cell transplant) and:
has received at least 2 prior lines of therapy for multiple myeloma, for example at least 2 prior lines of therapy including lenalidomide and a protease inhibitor, either sequentially or as part of a combined treatment regimen; and/or
is refractory (for example relapsed and refractory, or refractory) to the last line of therapy and/or to lenalidomide administered within 18 months prior to the treatment; and/or
is refractory (for example relapsed and refractory, or refractory) to at least an alyklator; and/or
is refractory (for example relapsed and refractory, or refractory) to at least an anti-CD38 antibody; and/or
is refractory (for example relapsed and refractory, or refractory) to at least an immunomodulatory drug (IMiDs); and/or
is refractory (e.g. refractory or relapsed-refractory) to lenalidomide, and in particular refractory (e.g. refractory or relapsed-refractory) to lenalidomide wherein lenalidomide was the last treatment that the patient received for multiple myeloma; and/or
is refractory to one or more (for example 1, 2, 3, 4 or 5 or more) drug from two of more classes of drugs selected from protease inhibitors (PIs), immunomodulatory drugs (IMiDs), alkylators and anti-CD38 antibody; and/or
is refractory (e.g. refractory or relapsed-refractory) to at least lenalidomide and 1, 2, 3 or 4 other drugs, for example at least one drug selected from protease inhibitor (PI), immunomodulatory drug (IMiD) alkylators and anti-CD38 antibody (or example, 2, 3 or 4 other drugs including at least one protease inhibitor (PI) and immunomodulatory drug (IMiD).

In such embodiments, the patient may have RRMM and/or the patient may be 75 years old or older.

The present invention is also especially beneficial for a patient having multiple myeloma who has not received a stem cell transplant, or has received a stem cell transplant that was at least 5 years ago, or has received a stem cell transplant and the disease subsequently progressed at least 36 months after the transplant, and/or is 75 years old or older (and especially a patient who has not received a stem cell transplant), and:
has received 2 prior lines of therapy; and/or
is alkylator refectory (refractory or relapsed refectory); and/or
is anti-CD38 antibody refectory (refractory or relapsed refectory); and/or
the last treatment for multiple myeloma that the patient received was a immunomodulatory drug (IMiDs), and in particular lenalidomide; and/or
is refractory (relapsed and refractory or refractory) to both the last line of therapy and to lenalidomide (>10 mg) administered within 18 months prior to treatment.

In such embodiments, the patient may have RRMM and/or the patient may be 75 years old or older.

A combination of melflufen, or a salt thereof, and dexamethasone for use according to the present invention is very useful in the treatment of refractory, relapsed and relapsed-refractory multiple myeloma, and more especially in the treatment of relapsed-refractory multiple myeloma. For example the methods and uses of the present invention comprising administering melflufen and dexamethasone, are useful for patients refractory (e.g. refractory or relapsed-refractory) to a protease inhibitor (PIs), immunomodulatory drug (IMiDs), alkylator or anti-CD38 antibody. It is especially useful in patients that are refractory (e.g. refractory or relapsed-refractory) to an alkylator, for example one or more of low dose melphalan, high dose melphalan and cyclophosphamide. It is also useful for patients refractory to one or more (for example 1, 2, 3, 4 or 5 or more) drug from two of more classes of drugs selected from protease inhibitors (PIs), immunomodulatory drugs (IMiDs), alkylators and anti-CD38 antibody. The methods and uses of the present invention comprising administering melflufen and dexamethasone, are also very especially useful in patients that are refractory (e.g. refractory or relapsed-refractory) to at least one immunomodulatory drug (IMiD), and more especially in patients that are refractory (e.g. refractory or relapsed-refractory) to at least the immunomodulatory drug lenalidomide; and more especially to at least lenalidomide and 1, 2, 3 or 4 other drugs, for example at least one drug selected from protease inhibitor (PI), immunomodulatory drug (IMiD) alkylators and anti-CD38 antibody. For example, 2, 3 or 4 other drugs including at least one protease inhibitor (PI) and immunomodulatory drug (IMiD). The methods and uses of the present invention comprising administering melflufen and dexamethasone, are also especially useful in patients that are refractory (e.g. refractory or relapsed-refractory) to at least pomalidomide and/or daratumumab.

As described above, the present invention provides a treatment for subpopulations of multiple myeloma patients. One subpopulation is multiple myeloma patients who have not had a stem cell transplant. For the avoidance of doubt, a “patient who has not received a stem cell transplant” is a patient who has never received a stem cell transplant during their lifetime. Therefore, prior to (or during) the treatment of the invention the patient has never received a stem cell transplant. Examples of stem cell transplants include autologous stem cell transplant, a tandem stem cell transplant, an allogeneic stem cell transplant, a donor lymphocyte infusion or a mini transplant. In exemplary embodiments of the invention, a patient who has not received a stem cell transplant is a patient who who has not received an autologous stem cell transplant.

A patient who has not received a stem cell transplant may have chosen not to receive that treatment, or may have been advised by a medical professional that they were not a suitable candidate for this type of treatment after individual assessment of their health. For example, stem cell transplant is usually not recommended for patients over age 65 or 70 unless they are in excellent physical health. Stem cell transplant is also not usually not recommended for a patient having underlying medical conditions such as heart disease and/or pulmonary disease. Stem cell transplant may also not be recommend depending on other factors, for example the type and the stage of the multiple myeloma, its aggressiveness and responsiveness to treatment. As there are many reasons why a multiple myeloma patient may not have had a stem cell transplant, based on choice or disease characterises, it is very surprising that this is a patient population that can benefit for a specific treatment. As shown in FIGS. 1 to 4, there are benefits across patient subgroups within the population of patients that have not had a stem cell transplant.

In certain preferred embodiments of the invention, the patient having multiple myeloma has not received a stem cell transplant and:
is at least 65, 70, 75 or 80 years old (preferably at least 75 or 80 years old); and/or
has cardiovascular disease; and/or
has pulmonary disease.

The present investors have also found that the treatment of the present invention for multiple myeloma patients that have not received a stem cell transplant is also especially beneficial in a patient that has not received a stem cell transplant and:
has a median body surface area (BSA) of ≤1.855 m²; and/or
has multiple myeloma with a Revised Multiple Myeloma International Staging System (R-ISS) of ISS grouping of I or II; and/or
is a high risk patient in view of the patient's cytogenetics; and/or
has impaired renal function (for example, a creatine clearance of less than 60 milliliters per minute (mL/min), or 60 to 90 mL/min; and in particular a creatine clearance of less than 60 mL/min.

The present inventors have also found that the treatment of the present invention for multiple myeloma patients that have not received a stem cell transplant is very especially beneficial in a patient that has not received a stem cell transplant and:
has RRMM; and/or
is refractory to lenalidomide (for example refractory to lenalidomide (for example ≥10 mg) administered within 18 months prior to treatment); and/or
has received at least 2 previous lines of therapy (for example 2, 3, or 4 previous lines of therapy).

The present inventors have also found that the treatment of the present invention for multiple myeloma patients that have not received a stem cell transplant is very especially beneficial in a patient that has not received a stem cell transplant and:
has RRMM; and
is refractory to lenalidomide (for example refractory to lenalidomide (for example >10 mg) administered within 18 months prior to treatment); and
has received at least 2 previous lines of therapy (for example 2, 3, or 4 previous lines of therapy).

As a treatment of the invention is for a patient that has not received a stem cell transplant, the methods and uses of the present invention may further comprise a step of determining if the patient has received a stem cell transplant, and if the patient has not received a stem cell transplant, administering melflufen, or a salt thereof, to a patient having multiple myeloma who has not received a stem cell transplant.

An ordinarily skilled physician can readily determine if a patient having multiple myeloma has received a stem cell transplant, for example by asking the patient and/or checking their medical records.

For example, the invention provides a method for the treatment or prophylaxis of multiple myeloma in a multiple myeloma patient, comprising determining if the patient has received a stem cell transplant, and if the patient has not received a stem cell transplant, administering melflufen, or a salt thereof, to a patient having multiple myeloma who has not received a stem cell transplant.

The invention further provides a method for the treatment or prophylaxis of multiple myeloma in a multiple myeloma patient, comprising determining if the patient has received a stem cell transplant, and if the patient has not received a stem cell transplant, administering melflufen, or a salt thereof, and dexamethasone to a patient having multiple myeloma who has not received a stem cell transplant. The present investors have also found that the treatment of the present invention for multiple myeloma patients that have not received a stem cell transplant is also especially beneficial in a patient who has not received a stem cell transplant and who is not eligible (also referred to herein as ‘not suitable’) for a stem cell transplant.

An ordinarily skilled physician can readily determine if a patient having multiple myeloma is suitable for a stem cell transplant after individual assessment of their health, for example considering the age, physical fitness, underlying medical conditions (such as heart disease and/or pulmonary disease) and other factors, for example the type and the stage of the multiple myeloma, its aggressiveness and responsiveness to treatment.

As such, the present invention may further comprise a step of determining if the patient is suitable for a stem cell transplant, and if the patient is not suitable for a stem cell transplant, administering melflufen, or a salt thereof, to a patient having multiple myeloma who has not received a stem cell transplant.

For example, the invention provides a method for the treatment or prophylaxis of multiple myeloma in a multiple myeloma patient, comprising determining if the patient is suitable for a stem cell transplant, and if the patient is not suitable for a stem cell transplant, administering melflufen, or a salt thereof, to a patient having multiple myeloma who has not received a stem cell transplant.

The invention further provides a method for the treatment or prophylaxis of multiple myeloma in a multiple myeloma patient, comprising determining if the patient is suitable for a stem cell transplant, and if the patient is not suitable for a stem cell transplant, administering melflufen, or a salt thereof, and dexamethasone to a patient having multiple myeloma who has not received a stem cell transplant.

The invention further provides a method for the treatment or prophylaxis of multiple myeloma in a multiple myeloma patient, comprising determining if the patient has received a stem cell transplant and determining if the patient is suitable for a stem cell transplant, and if the patient has not received a stem cell transplant and is not suitable for a stem cell transplant, administering melflufen, or a salt thereof, to a patient having multiple myeloma who has not received a stem cell transplant.

In certain embodiments, the invention provides a method for the treatment or prophylaxis of multiple myeloma in a multiple myeloma patient, comprising determining if the patient has received a stem cell transplant and if the patient is suitable for a stem cell transplant, and if the patient has not received a stem cell transplant and is not suitable for a stem cell transplant, administering melflufen, or a salt thereof, and dexamethasone to a patient having multiple myeloma who has not received a stem cell transplant.

The present invention further provides melflufen, or a salt thereof, for use in the treatment or prophylaxis of multiple myeloma in a patient having multiple myeloma who has not received a stem cell transplant and the patient is not suitable for a stem cell transplant. The present invention further provides melflufen, or a salt thereof, and dexamethasone for use in the treatment or prophylaxis of multiple myeloma in a patient having multiple myeloma, who has not received a stem cell transplant and who is not suitable for a stem cell transplant.

The present invention further provides a treatment for a subpopulation of multiple myeloma patients that have received a stem cell transplant that was at least 2.5 years ago, and preferably at least 5 years ago. For the avoidance of doubt, a “patient who has received a stem cell transplant that was at least X years ago” is a patient who has had a stem cell transplant in the past but has not received a stem cell transplant for at least X years before receiving a treatment according to the present invention. (For example, a “patient who has received a stem cell transplant that was at least 5 years ago” is a patient who has received a stem cell treatment in the past but who has not received a stem cell transplant for at least 5 years before receiving a treatment according to the present invention.) Therefore, for at least X years (for example 5 years) prior to a treatment of the invention the patient has not received a stem cell transplant, but they did receive a stem cell transplant more than X years (for example 5 years) in the past. As such, such a patient could alternatively be described as a patient “who has not received a stem cell transplant for at least X years”. For example, a patient who has received a stem cell transplant that was at least 5 years ago could alternatively be described as a patient who has not received a stem cell transplant for at least 5 years. Examples of stem cell transplants include autologous stem cell transplant, a tandem stem cell transplant, an allogeneic stem cell transplant, a donor lymphocyte infusion or a mini transplant. In exemplary embodiments of the invention, a patient who has received a stem cell transplant that was at least X years ago (for example at least 5 years ago) is a patient who has received an autologous stem cell transplant that was at least X years ago (for example at least 5 years ago).

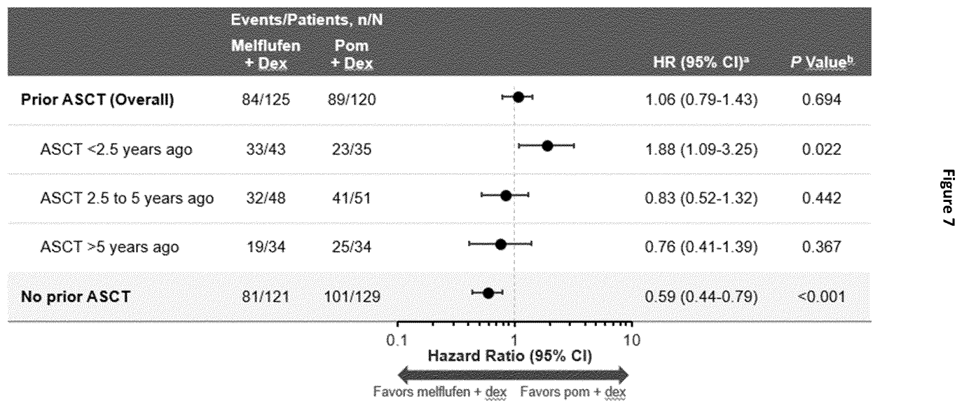

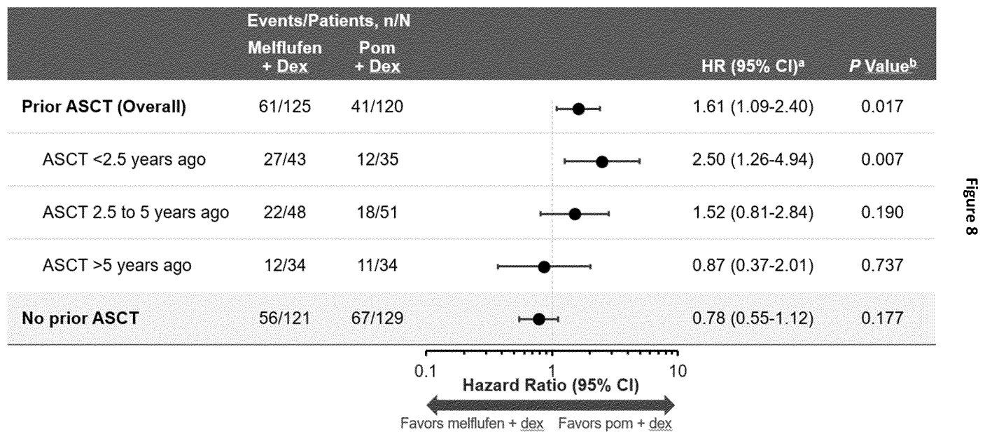

In certain embodiments, the present invention provides a treatment for a subpopulation of multiple myeloma patients who have received a stem cell transplant that was at least 2.5 years ago, at least 5 years ago, at least 6 years ago, at least 7 years ago, at least 8 years ago, at least 9 years ago, at least 10 years ago, at least 12 years ago or at least 15 years ago. In preferred embodiments, the present invention provides a treatment for a subpopulation of multiple myeloma patients who have received a stem cell transplant that was at least at least 5 years ago, at least 6 years ago, at least 7 years ago, at least 8 years ago, at least 9 years ago, at least 10 years ago, at least 12 years ago or at least 15 years ago. A patient who has received a stem cell transplant that was at least 5 years ago may have chosen not to receive a further stem cell transplant treatment in that time, or may have been advised by a medical professional that they were not a suitable candidate for this type of treatment after individual assessment of their health. For example, due to their age, underlying medical conditions (such as heart disease and/or pulmonary disease) or other factors, for example the type and the stage of the multiple myeloma, its aggressiveness and responsiveness to treatment. As shown in FIGS. 7 and 8 there are benefits across patient subgroups within the population of patients that have received a stem cell transplant that was at least 2.5 years ago, and especially at least 5 years ago, in particular compared to patients that have received a stem cell transplant in the previous 2.5 years.

In certain embodiments of the invention, the patient having multiple myeloma has received a stem cell transplant that was at least 5 years ago and:
is at least 65, 70, 75 or 80 years old (preferably at least 75 or 80 years old); and/or
has cardiovascular disease; and/or
has pulmonary disease.

In certain embodiments of the invention, the patient having multiple myeloma has received a stem cell transplant that was at least 5 years ago and:
has a median body surface area (BSA) of ≤1.855 m²; and/or
has multiple myeloma with a Revised Multiple Myeloma International Staging System (R-ISS) of ISS grouping of I or II; and/or
is a high risk patients in view of the patient's cytogenetics; and/or
has impaired renal function (for example, a creatine clearance of less than 60 milliliters per minute (mL/min), or 60 to 90 mL/min; and in particular a creatine clearance of less than 60 mL/min.

In certain embodiments of the invention, the patient having multiple myeloma has received a stem cell transplant that was at least 5 years ago and:
has RRMM; and/or (preferably and)
is refractory to lenalidomide (for example refractory to lenalidomide (for example ≥10 mg) administered within 18 months prior to treatment); and/or (preferably and)
has received at least 2 previous lines of therapy (for example 2, 3, or 4 previous lines of therapy).

As a treatment of the invention is for a patient that has received a stem cell transplant that was at least 5 years ago, the methods and uses of the present invention may further comprise a step of determining if the patient has received a stem cell transplant that was at least 5 years ago, and if the patient has received a stem cell transplant that was at least 5 years ago, administering melflufen, or a salt thereof, to the patient.

An ordinarily skilled physician can readily determine if a patient having multiple myeloma has received a stem cell transplant that was at least 5 years ago, for example by asking the patient and/or checking their medical records if they have received a stem cell transplant, and if so, if they had a stem cell transplant less than 5 years ago.

For example, the invention provides a method for the treatment or prophylaxis of multiple myeloma in a multiple myeloma patient, comprising determining if the patient has received a stem cell transplant that was at least 5 years ago and, if the patient has received a stem cell transplant that was at least 5 years ago, administering melflufen, or a salt thereof, to a patient having multiple myeloma who has received a stem cell transplant that was at least 5 years ago.

The invention further provides a method for the treatment or prophylaxis of multiple myeloma in a multiple myeloma patient, comprising determining if the patient has received a stem cell transplant that was at least 5 years ago, and if the patient has received a stem cell transplant that was at least 5 years ago, administering melflufen, or a salt thereof, and dexamethasone to a patient having multiple myeloma who has received a stem cell transplant that was at least 5 years ago.

The present investors have also found that the treatment of the present invention for multiple myeloma patients that have not received a stem cell transplant is also especially beneficial in a patient who has received a stem cell transplant that was at least 5 years ago and who is not eligible (also referred to herein as ‘not suitable’) for a (further) stem cell transplant.

As a treatment of the invention is for a patient who has received a stem cell transplant that was at least 5 years ago, the methods and uses of the present invention may further comprise a step of determining if the patient is suitable for a stem cell transplant, and if the patient is not suitable for a stem cell transplant, administering melflufen, or a salt thereof, to a patient having multiple myeloma who has received a stem cell transplant that was at least 5 years ago.

For example, the invention provides a method for the treatment or prophylaxis of multiple myeloma in a multiple myeloma patient, comprising determining if the patient is suitable for a stem cell transplant, and if the patient is not suitable for a stem cell transplant, administering melflufen, or a salt thereof, to a patient having multiple myeloma who has received a stem cell transplant that was at least 5 years ago.

The invention further provides a method for the treatment or prophylaxis of multiple myeloma in a multiple myeloma patient, comprising determining if the patient is suitable for a stem cell transplant, and if the patient is not suitable for a stem cell transplant, administering melflufen, or a salt thereof, and dexamethasone to a patient having multiple myeloma who has received a stem cell transplant that was at least 5 years ago.

The invention further provides a method for the treatment or prophylaxis of multiple myeloma in a multiple myeloma patient, comprising determining if the patient has received a stem cell transplant that was at least 5 years ago, and determining if the patient is suitable for a stem cell transplant, and if the patient has received a stem cell transplant that was at least 5 years ago and is not suitable for a stem cell transplant, administering melflufen, or a salt thereof, to a patient having multiple myeloma who has received a stem cell transplant that was at least 5 years ago.

In certain embodiments, the invention provides a method for the treatment or prophylaxis of multiple myeloma in a multiple myeloma patient, comprising determining if the patient has received a stem cell transplant that was at least 5 years ago, and if the patient is suitable for a stem cell transplant, and if the patient has received a stem cell transplant that was at least 5 years ago and is not suitable for a stem cell transplant, administering melflufen, or a salt thereof, and dexamethasone to a patient having multiple myeloma who has received a stem cell transplant that was at least 5 years ago.

The present invention further provides melflufen, or a salt thereof, for use in the treatment or prophylaxis of multiple myeloma in a patient having multiple myeloma who has received a stem cell transplant that was at least 5 years ago and the patient is not suitable for a stem cell transplant. The present invention further provides melflufen, or a salt thereof, and dexamethasone for use in the treatment or prophylaxis of multiple myeloma in a patient having multiple myeloma, who has received a stem cell transplant that was at least 5 years ago and who is not suitable for a stem cell transplant.

The present invention also provides a treatment for a subpopulation of multiple myeloma patients who are 75 years old or older. Such patients may also have not received a stem cell treatment, or have received a stem cell transplant that was at least 5 years ago. As discussed in the Results section of Example 1, as well as show in FIGS. 1 and 3, there are benefits of the treatment of the present invention across patient subgroups within the population of patients that are 75 years old or older.

In certain embodiments, the present invention provides a treatment for a subpopulation of multiple myeloma patients who are 78 years old or older, 80 years old or older, or 85 years old or older.

In certain embodiments of the invention, the patient having multiple myeloma is 75 years old or older and:
has not received a stem cell transplant, or has received a stem cell transplant that was at least 5 years ago (for example at least 6 years ago, at least 7 years ago, at least 8 years ago, at least 9 years ago, at least 10 years ago, at least 12 years ago or at least 15 years ago); and/or
has cardiovascular disease; and/or
has pulmonary disease.

In certain embodiments of the invention, the patient having multiple myeloma is 75 years old or older and:
has not received a stem cell transplant, or has received a stem cell transplant that was at least 5 years ago (for example at least 6 years ago, at least 7 years ago, at least 8 years ago, at least 9 years ago, at least 10 years ago, at least 12 years ago or at least 15 years ago); and/or
has a median body surface area (BSA) of ≤1.855 m²; and/or
has multiple myeloma with a Revised Multiple Myeloma International Staging System (R-ISS) of ISS grouping of I or II; and/or
is a high risk patients in view of the patient's cytogenetics; and/or has impaired renal function (for example, a creatine clearance of less than 60 milliliters per minute (mL/min), or 60 to 90 mL/min; and in particular a creatine clearance of less than 60 mL/min.

In certain embodiments of the invention, the patient having multiple myeloma is 75 years old or older and:
has RRMM; and/or (preferably and)
is refractory to lenalidomide (for example refractory to lenalidomide (for example ≥10 mg) administered within 18 months prior to treatment); and/or (preferably and)
has received at least 2 previous lines of therapy (for example 2, 3, or 4 previous lines of therapy).

In such embodiments, the patient may optionally have not received a stem cell transplant, or have received a stem cell transplant that was at least 5 years ago (for example at least 6 years ago, at least 7 years ago, at least 8 years ago, at least 9 years ago, at least 10 years ago, at least 12 years ago or at least 15 years ago).

As a treatment of the invention is for a patient that is 75 years old or older, the methods and uses of the present invention may further comprise a step of determining if the patient is 75 years old or older, and if the patient is 75 years old or older, administering melflufen, or a salt thereof, to a patient having multiple myeloma who is 75 years old or older.

An ordinarily skilled physician can readily determine if a patient having multiple myeloma is 75 years old or older, for example by asking the patient and/or checking their medical records.

For example, the invention provides a method for the treatment or prophylaxis of multiple myeloma in a multiple myeloma patient, comprising determining if the patient is 75 years old or older, and if the patient is 75 years old or older, administering melflufen, or a salt thereof, to a patient having multiple myeloma who is 75 years old or older.

The invention further provides a method for the treatment or prophylaxis of multiple myeloma in a multiple myeloma patient, comprising determining if the patient is 75 years old or older, and if the patient is 75 years old or older, administering melflufen, or a salt thereof, and dexamethasone to a patient having multiple myeloma who is 75 years old or older.

The present investors have also found that the treatment of the present invention for multiple myeloma patients that is 75 years old or older is also especially beneficial in a patient who is 75 years old or older and who is not eligible (also referred to herein as ‘not suitable’) for a stem cell transplant (and even more especially who has not received a stem cell transplant or has received a stem cell transplant that was at least 5 years ago). As a treatment of the invention is for a patient who is 75 years old or older, the methods and uses of the present invention may further comprise a step of determining if the patient is suitable for a stem cell transplant, and if the patient is not suitable for a stem cell transplant, administering melflufen, or a salt thereof, to a patient having multiple myeloma who has is 75 years old or older. For example, the invention provides a method for the treatment or prophylaxis of multiple myeloma in a multiple myeloma patient, comprising determining if the patient is suitable for a stem cell transplant, and if the patient is not suitable for a stem cell transplant, administering melflufen, or a salt thereof, to a patient having multiple myeloma who is 75 years old or older. The invention further provides a method for the treatment or prophylaxis of multiple myeloma in a multiple myeloma patient, comprising determining if the patient is suitable for a stem cell transplant, and if the patient is not suitable for a stem cell transplant, administering melflufen, or a salt thereof, and dexamethasone to a patient having multiple myeloma who is 75 years old or older.

The invention further provides a method for the treatment or prophylaxis of multiple myeloma in a multiple myeloma patient, comprising determining if the patient is 75 years old or older and determining if the patient is suitable for a stem cell transplant, and if the patient is 75 years old or older and is not suitable for a stem cell transplant, administering melflufen, or a salt thereof, to a patient having multiple myeloma who is 75 years old or older.

In certain embodiments, the invention provides a method for the treatment or prophylaxis of multiple myeloma in a multiple myeloma patient, comprising determining if the patient is 75 years old or older and if the patient is suitable for a stem cell transplant, and if the patient is 75 years old or older and is not suitable for a stem cell transplant, administering melflufen, or a salt thereof, and dexamethasone to a patient having multiple myeloma who is 75 years old or older.

The present invention further provides melflufen, or a salt thereof, for use in the treatment or prophylaxis of multiple myeloma in a patient having multiple myeloma who is 75 years old or older and the patient is not suitable for a stem cell transplant. The present invention further provides melflufen, or a salt thereof, and dexamethasone for use in the treatment or prophylaxis of multiple myeloma in a patient having multiple myeloma, who is 75 years old or older and who is not suitable for a stem cell transplant.

The invention further provides a method for the treatment or prophylaxis of multiple myeloma in a multiple myeloma patient, comprising determining if the patient has received a stem cell transplant and the disease subsequently progressed at least 36 months after the transplant and, if the patient had received a stem cell transplant and the disease subsequently progressed at least 36 months after the transplant, administering melflufen or a salt thereof to the patient.

The invention further provides a method for the treatment or prophylaxis of multiple myeloma in a multiple myeloma patient, comprising determining if the patient has received a stem cell transplant and the disease subsequently progressed at least 36 months after the transplant and, if the patient had received a stem cell transplant and the disease subsequently progressed at least 36 months after the transplant, administering melflufen or a salt thereof, and dexamethasone to the patient.

In such uses and methods of treatment for a patient having multiple myeloma who is 75 years old or older, the patient may also be a patient having multiple myeloma who has not received a stem cell transplant, or be a patient having multiple myeloma who has received a stem cell transplant that was at least 5 years ago. As such, in uses and methods of treatment for a patient having multiple myeloma who is 75 years old or older, and who has not received a stem cell transplant, the methods and uses of the present invention may further comprise a step of determining if the patient is has received a stem cell transplant, and if the patient has not received for a stem cell transplant, administering melflufen, or a salt thereof, (and optionally dexamethasone) to a patient having multiple myeloma who is 75 years old or older and who has not received a stem cell transplant. In uses and methods of treatment for a patient having multiple myeloma who is 75 years old or older, and who has received a stem cell transplant that was at least 5 years ago, the methods and uses of the present invention may further comprise a step of determining if the patient is has received a stem cell transplant that was at least 5 years ago, and if the patient has received a stem cell transplant that was at least 5 years ago, administering melflufen, or a salt thereof, (and optionally dexamethasone) to a patient having multiple myeloma who is 75 years old or older and who has received a stem cell transplant that was at least 5 years ago.

The following clauses further define embodiments of the present invention:

§1. Melflufen, or a salt thereof, and dexamethasone for use in the treatment or prophylaxis of multiple myeloma in a patient having multiple myeloma who
has not received a stem cell transplant; or
has received a stem cell transplant that was at least 5 years ago; or
is 75 years old or older; or
has not received a stem cell transplant and is 75 years old or older, or has received a stem cell transplant that was at least 5 years ago and is 75 years old or older; or
has received a stem cell transplant and the disease subsequently progressed at least 36 months after the transplant.

§2. Melflufen, or a salt thereof, and dexamethasone for use as defined in clause 1, wherein the melflufen is administered in a dose of about 1 to 150 mg (excluding the mass of any counterion), for example a dose of 1 to 50 mg (excluding the mass of any counterion) (for example 1, 5, 10, 15, 20, 25, 30, 35, 40, 45 or 50 mg).

§3. Melflufen, or a salt thereof, and dexamethasone for use as defined in clause 1 or 2, wherein the dexamethasone is administered in a dose of about 5 mg to 100 mg, more preferably 10 mg to 80 mg, and most preferably 20 mg to 60 mg (for example 40 mg or 20 mg).

§4. Melflufen, or a salt thereof, for use as defined in any one of clauses 1 to 3, wherein a dose of melflufen is administered on day 1 of a cycle of 1 to 42 days (for example 21 to 35 days, and in particular 21, 28, 29, 30 or 35 days).

§5. Melflufen, or a salt thereof, and dexamethasone for use as defined in any one of clauses 1 to 4, wherein a dose of melflufen (excluding the mass of any salt) is administered as a parenteral dosage at an infusion rate of 0.3 to 1.8 mg/min (for example as a parenteral dosage at an infusion rate of 1.1 to 1.8 mg/min).

§6. Melflufen, or a salt thereof, and dexamethasone for use as defined in any one of clauses 1 to 5, wherein the melflufen, or salt thereof, is administered simultaneously, sequentially or separately with the dexamethasone (for example a dose of 20 mg or 40 mg of dexamethasone).

§7. Melflufen, or a salt thereof, and dexamethasone for use as defined in any one of clauses 1 to 5, wherein the melflufen, or salt thereof, is administered simultaneously, sequentially or separately with one or more further therapeutic agent(s), for example wherein the one or more further therapeutic agent(s) is selected from steroids (e.g. prednisone), IMiDs (e.g. thalidomide, lenalidomide and pomalidomide), PIs (e.g. bortezomib, carfilzomib, and ixazomib), histone deacetylase (HDAC) inhibitors (e.g. panobinostat), conventional chemotherapy (e.g. melphalan, cyclophosphamide, doxorubicin, bendamustine), anti-CD38 antibodies (daratumumab) and anti-SLAMF7 antibodies (elotuzumab); and preferably the one or more further therapeutic agent(s) selected from daratumumab and bortezomib;.

§8. A pharmaceutical formulation comprising melflufen, or a salt thereof, and a pharmaceutical formulation comprising dexamethasone for use as defined in any one of clauses 1 to 7.

§9. A method for the treatment or prophylaxis of multiple myeloma, comprising the step of administering melflufen, or a salt thereof, and dexamethasone to a patient having multiple myeloma who
has not received a stem cell transplant; or
has received a stem cell transplant that was at least 5 years ago; or
who is 75 years old or older; or
has not received a stem cell transplant and is 75 years old or older, or has received a stem cell transplant that was at least 5 years ago and is 75 years old or older.

§10. The method of clause 9 wherein the patient is additionally administered simultaneously, sequentially or separately from melflufen, or a salt there, one or more further therapeutic agent(s) which is selected from steroids (e.g. prednisone and dexamethasone), IMiDs (e.g. thalidomide, lenalidomide and pomalidomide), PIs (e.g. bortezomib, carfilzomib, and ixazomib), histone deacetylase (HDAC) inhibitors (e.g. panobinostat), conventional chemotherapy (e.g. melphalan, cyclophosphamide, doxorubicin, bendamustine), anti-CD38 antibodies (daratumumab) and anti-SLAMF7 antibodies (elotuzumab); or selected from antibodies against the B-cell maturation antigen (belantamab), inhibitors of nuclear export (selinexor) and autologous chimeric antigen receptor (CAR) T-cell therapy directed against the B-cell maturation antigen (ciltacabtagene) and preferably the one or more further therapeutic agent(s) selected from daratumumab and bortezomib.

§11. The method of clause 9 or 10, wherein the method comprises determining if the patient has received for a stem cell transplant and if the patient has not received a stem cell transplant, administering melflufen, or a salt thereof, to the patient; or determining if the patient has received a stem cell transplant that was at least 5 years ago and if the patient has received a stem cell transplant that was at least 5 years ago, administering melflufen, or a salt thereof, to the patient; and/or determining if the patient is 75 years old or older, and if the patient is 75 years old or older, administering melflufen, or a salt thereof, to the patient.

§12. The method of clause 9, 10 or 11, wherein the method comprises determining if the patient is suitable for a stem cell transplant and if the patient is not suitable for a stem cell transplant, administering melflufen, or a salt thereof, to the patient.

§13. The use of melflufen, or a salt thereof, and dexamethasone for the manufacture of a medicament for the treatment of multiple myeloma in a patient having multiple myeloma who
has not received a stem cell transplant; or
has received a stem cell transplant that was at least 5 years ago; or
is 75 years old or older; or
has not received a stem cell transplant and is 75 years old or older, or has received a stem cell transplant that was at least 5 years ago and is 75 years old or older; or
has received a stem cell transplant and the disease subsequently progressed at least 36 months after the transplant.

§14. A kit comprising melflufen, dexamethasone and one or more further therapeutic agent(s) selected from steroids (e.g. prednisone), IMiDs (e.g. thalidomide, lenalidomide and pomalidomide), PIs (e.g. bortezomib, carfilzomib, and ixazomib), histone deacetylase (HDAC) inhibitors (e.g. panobinostat), conventional chemotherapy (e.g. melphalan, cyclophosphamide, doxorubicin, bendamustine), anti-CD38 antibodies (daratumumab) and anti-SLAMF7 antibodies (elotuzumab); or selected from antibodies against the B-cell maturation antigen (belantamab), inhibitors of nuclear export (selinexor) and autologous chimeric antigen receptor (CAR) T-cell therapy directed against the B-cell maturation antigen (ciltacabtagene); and preferably the one or more further therapeutic agent(s) selected from daratumumab and bortezomib;

for use in the treatment or prophylaxis of multiple myeloma in a patient as defined in any one of clauses 1 to 7.

§15. The melflufen, or a salt thereof, for use as defined in any one of clauses 1 to 7, the pharmaceutical formulation as defined in clause 8, the method as defined in clause 9 to 12, the use as defined in clause 13, or the kit as defined in clause 14, wherein the multiple myeloma patient:
has received at least 2 prior lines of therapy for multiple myeloma, for example at least 2 prior lines of therapy including lenalidomide and a protease inhibitor, either sequentially or as part of a combined treatment regimen; and/or
is refractory (for example relapsed and refractory, or refractory) to the last line of therapy and/or to lenalidomide administered within 18 months prior to the treatment; and/or
is refractory (for example relapsed and refractory, or refractory) to at least an alyklator; and/or
is refractory (for example relapsed and refractory, or refractory) to at least an anti-CD38 antibody; and/or
is refractory (for example relapsed and refractory, or refractory) to at least an immunomodulatory drug (IMiDs); and/or
is refractory (e.g. refractory or relapsed-refractory) to lenalidomide, and in particular refractory (e.g. refractory or relapsed-refractory) to lenalidomide wherein lenalidomide was the last treatment that the patient received for multiple myeloma; and/or
is refractory to one or more (for example 1, 2, 3, 4 or 5 or more) drug from two of more classes of drugs selected from protease inhibitors (PIs), immunomodulatory drugs (IMiDs), alkylators and anti-CD38 antibody; and/or
is refractory (e.g. refractory or relapsed-refractory) to at least lenalidomide and 1, 2, 3 or 4 other drugs, for example at least one drug selected from protease inhibitor (PI), immunomodulatory drug (IMiD) alkylators and anti-CD38 antibody (or example, 2, 3 or 4 other drugs including at least one protease inhibitor (PI) and immunomodulatory drug (IMiD); and/or
is refractory (e.g. refractory or relapsed-refractory) to at least pomalidomide and/or daratumumab; and/or has RRMM.

§16. The melflufen, or a salt thereof, for use as defined in any one of clauses 1 to 7 or 15, the pharmaceutical formulation as defined in clause 8 or 15, the method as defined in clause 9 to 12 or 15, the use as defined in clause 13 or 15, or the kit as defined in clause 14 or 15, wherein the multiple myeloma patient:
is at least 65, 70, 75 or 80 years old; and/or
has cardiovascular disease; and/or
has pulmonary disease.

§17. The melflufen, or a salt thereof, for use as defined in any one of clauses 1 to 7 or 15 to 16, the pharmaceutical formulation as defined in clause 8 or 15 to 16, the method as defined in clause 9 to 12 or 15 to 16, the use as defined in clause 13 or 15 to 16, or the kit as defined in clause 14 to 16, wherein the multiple myeloma patient is not suitable for a stem cell transplant.

§18. The melflufen, or a salt thereof, for use as defined in any one of clauses 1 to 7 or 15 to 17, the pharmaceutical formulation as defined in clause 8 or 15 to 17, the method as defined in clause 9 to 12 or 15 to 17, the use as defined in clause 13 or 15 to 17, or the kit as defined in clause 14 to 17, wherein the multiple myeloma patient:
has a median body surface area (BSA) of ≤1.855 m²; and/or
has multiple myeloma with a Revised Multiple Myeloma International Staging System (R-ISS) of ISS grouping of I or II; and/or
is a high risk patients in view of the patient's cytogenetics; and/or
has impaired kidney function (for example, a creatine clearance of less than 60 milliliters per minute (mL/min), or 60 to 90 mL/min; and in particular a creatine clearance of less than 60 mL/min.

§19. The melflufen, or a salt thereof, for use as defined in any one of clauses 1 to 7 or 15 to 18, the pharmaceutical formulation as defined in clause 8 or 15 to 18, the method as defined in clause 9 to 12 or 15 to 18, the use as defined in clause 13 or 15 to 18, or the kit as defined in clause 14 to 18, wherein the multiple myeloma patient:
has RRMM; and/or (preferably and)
is refractory to lenalidomide (for example refractory to lenalidomide (for example ≥10 mg) administered within 18 months prior to treatment); and/or (preferably and)
has received at least 2 previous lines of therapy (for example 2, 3, or 4 previous lines of therapy).

§20. The melflufen, or a salt thereof, for use as defined in any one of clauses 1 to 7 or 15 to 19, the pharmaceutical formulation as defined in clause 8 or 15 to 19, the method as defined in clause 9 to 12 or 15 to 19, the use as defined in clause 13 or 15 to 19, or the kit as defined in clause 14 to 19, wherein the multiple myeloma patient has not received a stem cell transplant.

The following Example illustrates the invention.

### EXAMPLE 1

### Study Description

Brief Summary: A randomized, controlled, open-label, Phase 3 multicenter study which enrolled patients with RRMM following 2-4 lines of prior therapy and who were refractory to both the last line of therapy and to lenalidomide (≥10 mg) administered within 18 months prior to randomization as demonstrated by disease progression on or within 60 days of completion of the last dose of lenalidomide. Patients received either melflufen+dexamethasone or pomalidomide+dexamethasone.

### Detailed Description

A randomized, controlled, open-label, Phase 3 multicenter study which enrolled patients with RRMM following 2-4 lines of prior therapy and who are refractory to both the last line of therapy and to lenalidomide as demonstrated by disease progression on or within 60 days of completion of the last dose of lenalidomide. Patients were randomized to either one of two arms (see Table 1 below):
Arm A: Melphalan flufenamide (Melflufen) 40 mg on Day 1 and dexamethasone 40 mg on Days 1, 8, 15 and 22 of each 28-day cycle.
Arm B: Pomalidomide 4 mg daily on Days 1 to 21 and dexamethasone 40 mg on Days 1, 8, 15 and 22 of each 28-day cycle.

Patients ∝75 years of age had a reduced dose of dexamethasone of 20 mg on Days 1, 8, 15 and 22 for both Arm A and Arm B. Patients received treatment until such time as there is documented disease progression, unacceptable toxicity or the patient/treating physician determines it is not in the patient's best interest to continue. Dose modifications and delays in therapy were implemented based on patient tolerability as detailed in the protocol. In the event of a cycle delay, unrelated to dexamethasone toxicity, it was recommended to continue dexamethasone weekly.

### Study Design

Study Type: Interventional (Clinical Trial)
Actual Enrollment: 495 participants
Allocation: Randomized
Intervention Model: Parallel Assignment
Masking: Single (Outcomes Assessor)
Primary Purpose: Treatment
Official Title: A Randomized, Controlled, Open-label, Phase 3 Study of Melflufen/Dexamethasone Compared With Pomalidomide/Dexamethasone for Patients With Relapsed Refractory Multiple Myeloma Who Are Refractory to Lenalidomide

### Arms and Interventions

TABLE 1

| Arm | Intervention/treatment |
|---|---|
| Experimental: Arm A: | Drug: Melflufen |
| Melflufen + Dexamethasone | Intravenous infusion |
| Melflufen 40 mg i.v. on Day 1 and | Other Name: Melphalan |
| dexamethasone 40 mg on Days 1, 8, | Flufenamide |
| 15 and 22 of each 28-day cycle. | Drug: Dexamethasone |
| Patients are to be treated until | Oral tablets |
| confirmed progression, unacceptable | Other Names: |
| toxicity or the patient or investigator | Dex |
| decides it is not in the patient's | Fortecortin |
| best interest to continue. | Decadron |
| Active Comparator: Arm B: | Drug: Pomalidomide |
| Pomalidomide + Dexamethasone | Oral capsules |
| Pomalidomide 4 mg orally daily on Days 1 | Other Names: |
| to 21 and dexamethasone 40 mg on Days 1, | Pomalyst |
| 8, 15 and 22 of each 28-day cycle. | Imnovid |
| Patients are to be treated until | Drug: Dexamethasone |
| confirmed progression, unacceptable | Oral tablets |
| toxicity or the patient or investigator | Other Names: |
| decides it is not in the patient's | Dex |
| best interest to continue. | Fortecortin |
| | Decadron |

### Primary Outcome Measures

1. Progression Free Survival (PFS) [Time Frame: From randomization to time of progression, or, if no progression, 24 months after end of treatment]: To compare the PFS of melflufen plus dexamethasone (Arm A) versus pomalidomide plus dexamethasone (Arm B) as assessed by the Independent Review Committee (IRC) according to the International Myeloma Working Group Uniform Response Criteria (IMWG-URC)

### Secondary Outcome Measures

1. Overall Response Rate (ORR) [Time Frame: From randomization until best response achieved before confirmed progression, or if no progression, 24 months after end of treatment]:To assess and compare the ORR in Arm A versus Arm B
2. Duration of Response (DOR) [Time Frame: From first evidence of response until confirmed progression, or if no progression, 24 months after end of treatment]: To assess and compare the DOR in Arm A versus Arm B
3. Overall Survival (OS) [Time Frame: From randomization until end of study (2 years after confirmed progression)]: To assess and compare OS in Arm A versus Arm B
4. Safety and Tolerability: Number of patients with treatment-emergent adverse events, including clinical laboratory and vital signs abnormalities, as assessed by CTCAE v4.0 [Time Frame: From start of dosing until 30 days after last dose]: To assess and compare safety and tolerability in Arm A versus Arm B. Number of patients with treatment-emergent adverse events, including clinical laboratory and vital signs abnormalities, as assessed by CTCAE v4.0 will be presented. No formal statistical analysis will be performed for safety endpoints.

### Eligibility Criteria

Ages Eligible for Study: 18 Years and older (Adult, Older Adult)
Sexes Eligible for Study: All
Accepts Healthy Volunteers: No

### Inclusion Criteria:

1. Male or female, age 18 years or older
2. A prior diagnosis of multiple myeloma with documented disease progression requiring further treatment at time of screening
3. Measurable disease defined as any of the following:
   Serum monoclonal protein ≥0.5 g/dl by protein electrophoresis.
   ≥200 mg/24 hours of monoclonal protein in the urine on 24-hour electrophoresis
   Serum free light chain ≥10 mg/dl AND abnormal serum kappa to lambda free light chain ratio
4. Received 2-4 prior lines of therapy, including lenalidomide and a PI, either sequential or in the same line, and is refractory (relapsed and refractory or refractory) to both the last line of therapy and to lenalidomide (≥10 mg) administered within 18 months prior to randomization. Refractory to lenalidomide is defined as progression while on lenalidomide therapy or within 60 days of last dose, following at least 2 cycles of lenalidomide with at least 14 doses of lenalidomide per cycle
5. Life expectancy of ≥6 months
6. Eastern Cooperative Oncology Group (ECOG) performance status ≤2
7. Females of child bearing potential (FCBP) must have a negative serum or urine pregnancy test prior to start of treatment. Participants must agree to ongoing pregnancy testing. All patients must be willing to comply with all requirements of the USA pomalidomide Risk Evaluation and Mitigation Strategy (REMS) program or the pomalidomide Pregnancy Prevention Plan (PPP)
8. Ability to understand the purpose and risks of the study and provide signed and dated informed consent
9. 12-lead Electrocardiogram (ECG) with QT interval calculated by Fridericia Formula (QTcF) interval of ≤470 msec Fridericia Formula
10. The following laboratory results must be met during screening and also immediately before study drug administration on Cycle 1 Day 1:
   Absolute neutrophil count (ANC) ≥1,000 cells/mm3 (1.0×109/L)
   Platelet count ≥75,000 cells/mm3 (75×109/L)
   Hemoglobin ≥8.0 g/dl
   Total Bilirubin ≤1.5 x upper limit of normal (ULN), or patients diagnosed with Gilberts syndrome, that have been reviewed and approved by the medical monitor
   Aspartate transaminase (AST/SGOT) and alanine transaminase (ALT/SGPT) ≤3.0 x ULN
   Renal function: Estimated creatinine clearance by Cockcroft-Gault formula ≥45 ml/min
11. Must be able to take antithrombotic prophylaxis
12. Must have, or be willing to have an acceptable central catheter. (Port a cath, peripherally inserted central catheter [PICC-line], or central venous catheter) (Insertion only required if randomized to Arm A)

### Exclusion Criteria:

1. Primary refractory disease (i.e. never responded (≥MR) to any prior therapy)
2. Evidence of mucosal or internal bleeding or platelet transfusion refractory
3. Any medical conditions that, in the Investigator's opinion, would impose excessive risk to the patient or would adversely affect his/her participating in this study
4. Prior exposure to pomalidomide
5. Known intolerance to IMiDs
6. Known active infection requiring parenteral or oral anti-infective treatment within 14 days of randomization
7. Other malignancy diagnosed or requiring treatment within the past 3 years with the exception of adequately treated basal cell carcinoma, squamous cell skin cancer, carcinoma in-situ of the cervix or breast or very low and low risk prostate cancer in active surveillance
8. Pregnant or breast-feeding females
9. Serious psychiatric illness, active alcoholism, or drug addiction that may hinder or confuse compliance or follow-up evaluation
10. Known human immunodeficiency virus or active hepatitis C viral infection
11. Active hepatitis B viral infection (defined as HBsAg+)
   Patients with prior hepatitis B vaccine are permitted (defined as HBsAg−, Anti-HBs+, Anti-HBc-)
   Non-active hepatitis B (HBsAg−, Anti-HBS+, Anti-HBC+) may be enrolled at the discretion of the investigator after consideration of risk of reactivation
12. Concurrent symptomatic amyloidosis or plasma cell leukemia
13. POEMS syndrome
14. Previous cytotoxic therapies, including cytotoxic investigational agents, for multiple myeloma within 3 weeks (6 weeks for nitrosoureas) prior to randomization. IMiDs, PIs and or corticosteroids within 2 weeks prior to randomization. Other investigational therapies and monoclonal antibodies within 4 weeks of randomization. Prednisone up to but no more than 10 mg orally q.d. or its equivalent for symptom management of comorbid conditions is permitted but dose should be stable for at least 7 days prior to randomization
15. Residual side effects to previous therapy >grade 1 prior to randomization (Alopecia any grade and/or neuropathy grade 2 without pain are permitted)
16. Prior peripheral stem cell transplant within 12 weeks of randomization
17. Prior allogeneic stem cell transplantation with active graft-versus-host-disease.
18. Prior major surgical procedure or radiation therapy within 4 weeks of the randomization
19. Known intolerance to steroid therapy

### Results

Table 2, below, summarises some of the characteristics of the patients included in the Example 1 study.

TABLE 2

| | Melflufen + | Pom + |
|---|---|---|
| | Dex | Dex |
| Characteristics | (N = 246) | (N = 249) |
| Age, median (IQR), years | 68 (60-72) | 68 (61-72) |
| <65 years, n (%) | 96 (39) | 85 (34) |
| 65 to <75 years, n (%) | 113 (46) | 125 (50) |
| ≥75 years, n (%) | 37 (15) | 39 (16) |
| Male sex, n (%) | 139 (57) | 140 (56) |
| ECOG PS (0/1/2), % | 37/53/11 | 37/55/8 |
| ISS score (I/II/III) | 48/38/13 | 50/38/12 |
| at study entry, % | | |
| High-risk cytogenetics | 83 (34) | 86 (35) |
| at study entry^{a} | | |
| EMD at study entry | 31 (13) | 31 (12) |
| Previous lines of therapy, | 3 (2-3) | 3 (2-3) |
| median (IQR) | | |
| 2 vs 3 or 4, % | 46/54 | 45/55 |
| Previous ASCT, n (%) | 125 (51) | 120 (48) |
| Refractory to previous line | | |
| of therapy, n (%): | | |
| Alkylator | 78 (32) | 75 (30) |
| Anti-CD38 monoclonal | 48 (20) | 39 (16) |
| antibody | | |
| Lenalidomide | 245 (>99) | 248 (>99) |
| Lenalidomide in last | 213 (87) | 217 (87) |
| line of therapy | | |
| Proteasome inhibitor | 163 (66) | 163 (65) |
| Triple-class-refractory | 39 (16) | 30 (12) |
| disease^{b} | | |
| Last line of therapy^{c} | 245 (>99) | 247 (99) |

ASCT, autologous stem cell transplant; dex, dexamethasone; ECOG, Eastern Cooperative Oncology Group; EMD, extramedullary disease; IQR, interquartile range; ISS, International Staging System; melflufen, melphalan flufenamide; pom, pomalidomide; PS, performance status.
^{a}Defined as t(4; 14), t(14; 16), t(14; 20), del(17p), gain(1q21), or gain 1q(+1q) by fluorescence in situ hybridization.
^{b}Refractory to ≥1 immunomodulatory drug, ≥1 proteasome inhibitor, and ≥1 anti-CD38 monoclonal antibody.
^{c}Failure to achieve at least a minimal response or progression on therapy within 60 days of the last dose of treatment.

PFS and OS Forest plots showing hazard ratios for the patient group that had not received a stem cell transplant (n=121 for melflufen+dexamethasone subgroup; n=129 for the pomalidomide+dexamethasone subgroup) are shown in FIGS. 1 to 4. The hazard ratio is a measure of the relative risk of an event at each time point during follow-up when receiving melflufen in relation to pomalidomide. A value below 1 indicates a better treatment effect for melflufen, and a value above 1 indicates a better treatment effect for pomalidomide.

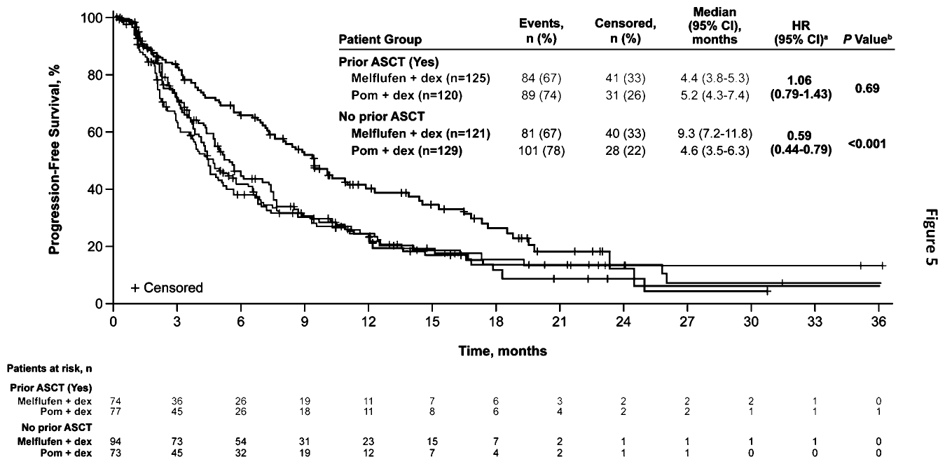

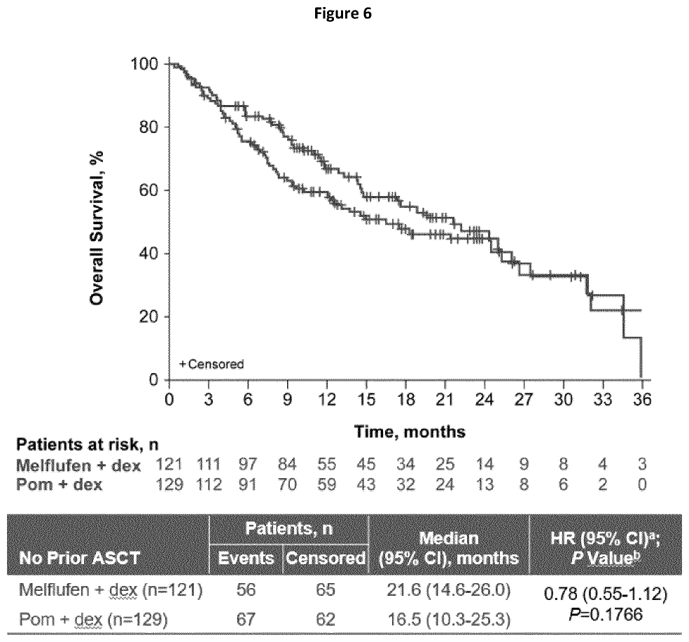

FIG. 5 shows a graph of PFS(%) over time for patients that had not received a stem cell transplant (n=121 for melflufen+dexamethasone subgroup; n=129 for the pomalidomide+dexamethasone subgroup), and those who had received a stem cell transplant (n=125 for melflufen+dexamethasone subgroup; n=120 for the pomalidomide+dexamethasone subgroup). The data in the graph are also presented as simple numbers underneath the graph. The top line in the graph shows the results for the melflufen+dexamethasone subgroup that had not received a stem cell transplant. FIG. 6 shows a graph of OS (%) over time for patients that had not received a stem cell transplant (n=121 for melflufen+dexamethasone subgroup; n=129 for the pomalidomide+dexamethasone subgroup). Again, the data in the graph are also presented as simple numbers underneath the graph. The top line in the graph shows the results for the melflufen+dexamethasone subgroup that had not received a stem cell transplant.

As can be seen from FIGS. 1 to 6, melflufen+dexamethasone provided a significantly better treatment effect in terms of PFS and OS compared to pomalidomide+dexamethasone for patients who had not previously received a stem cell transplant. Patients with multiple myeloma in the trial who had not received a stem cell transplant as a past treatment and who received melflufen (n=121) had a median PFS of 9.3 months, whereas patients with multiple myeloma who had not received a stem cell transplant as a past treatment and who received pomalidomide (n=129) had a median PFS of 4.6 months. Additionally, patients with multiple myeloma in the trial who had not received a stem cell transplant as a past treatment and who received melflufen (n=121) had a median OS of 21.6 months, whereas patients with multiple myeloma who had not received a stem cell transplant as a past treatment and who received pomalidomide (n=129) had a median OS of 16.5 months. As shown in FIGS. 1 to 4, these improved treatment effects of melflufen+dexamethasone compared to pomalidomide+dexamethasone were consistently present for demographic and disease characteristic subgroups within the subgroup of patients that had not received a stem cell transplant as a past treatment.

FIGS. 7 and 8 show tables of PFS hazard ratios and events (FIG. 7), and OS hazard ratios and events (FIG. 8), for patients in Example 1 that had not had a stem cell transplant and were treated with melflufen+dexamethasone (n=121) or pomalidomide+dexamethasone (n=129), or who were treated with melflufen+dexamethasone and had received a stem cell transplant less than 2.5 years ago (n=43), 2.5 to 5 years ago (n=48), or more than 5 years ago (n=34), or who were treated with pomalidomide+dexamethasone and had received a stem cell transplant less than 2.5 years ago (n=35), 2.5 to 5 years ago (n=51), or more than 5 years ago (n=34).

As can be seen from FIGS. 7 and 8, melflufen+dexamethasone provided a significantly better treatment effect in terms of PFS and OS compared to pomalidomide+dexamethasone for patients who had not received a stem cell transplant (PFS hazard ratio=0.59; OS hazard ratio=0.78), or who had received a stem cell transplant that was at least 5 years ago (PFS hazard ratio=0.73; OS hazard ratio=0.87). Melflufen+dexamethasone also provided a better treatment effect in terms of PFS compared to pomalidomide+dexamethasone for patients who have received a stem cell transplant 2.5 to 5 years ago (hazard ratio=0.83).

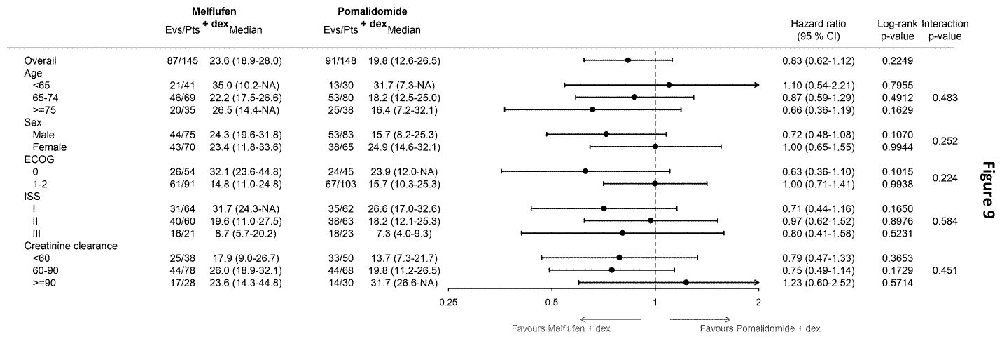

In the data shown in FIGS. 9 and 10, patients who had received a prior stem cell transplant and then suffered progression within 36 months are excluded from the analysis. That is to say that the figures represent patients who had not had a prior stem cell transplant, or who had had a prior stem cell transplant and then had disease progression more than 36 months afterwards. As can be seen from FIGS. 9 and 10, melflufen+dexamethasone provided a significantly better treatment effect in terms of OS (23.6 months) compared to pomalidomide+dexamethasone (19.8 months) for patients who are not in the group of patients that received a stem cell transplant and then suffered progression within 36 months (hazard ratio=0.83).

Finally, the present inventors surprisingly found that patients with multiple myeloma in the trial who were 75 or over and received melflufen had a median PFS of 9.4 months, whereas patients with multiple myeloma who had not received a stem cell transplant as a past treatment and who received pomalidomide had a median PFS of 4.6 months. Additionally, the inventors found that patients with multiple myeloma in the trial who were 75 or over and received melflufen had a median overall survival (OS) of 21.6 months, whereas patients with multiple myeloma who had not received a stem cell transplant as a past treatment and who received pomalidomide had a median OS of 8.3 months. A number of patients in that age range that were part of the Example 1 study had not previously received a stem cell transplant. It was also the case that a number of patients in that age range that were part of the Example 1 study had previously received a stem cell transplant and then had disease progression more than 36 months afterwards. In both of those sub-sets the improvement effect was very pronounced. Therefore, the results of the Example show that melflufen treatment was especially beneficial effects in patients of 75 years old or older, and in particular those of that age who has previously not received a stem cell transplant, and also in those of that age who had previously received a stem cell transplant and then had disease progression more than 36 months afterwards.

1. Melflufen, or a salt thereof, for use in the treatment or prophylaxis of multiple myeloma in a patient having multiple myeloma who
has not received a stem cell transplant; or
has received a stem cell transplant that was at least 5 years ago; or
is 75 years old or older; or
has not received a stem cell transplant and is 75 years old or older, or has received a stem cell transplant that was at least 5 years ago and is 75 years old or older; or
has received a stem cell transplant and the disease subsequently progressed at least 36 months after the transplant.

2. Melflufen, or a salt thereof, for use as claimed in claim 1, wherein the melflufen is administered in a dose of about 1 to 150 mg (excluding the mass of any counterion).

3. Melflufen, or a salt thereof, for use as claimed in claim 2, wherein the melflufen is administered in a dose of 1 to 50 mg (excluding the mass of any counterion) (for example 1, 5, 10, 15, 20, 25, 30, 35, 40, 45 or 50 mg).

4. Melflufen, or a salt thereof, for use as claimed in any one of claims 1 to 3, wherein a dose of melflufen is administered on day 1 of a cycle of 1 to 42 days (for example 21 to 35 days, and in particular 21, 28, 29, 30 or 35 days).

5. Melflufen, or a salt thereof, for use as claimed in any one of claims 1 to 4, wherein a dose of melflufen (excluding the mass of any salt) is administered as a parenteral dosage at an infusion rate of 0.3 to 1.8 mg/min (for example as a parenteral dosage at an infusion rate of 1.1 to 1.8 mg/min).

6. Melflufen, or a salt thereof, for use as claimed in any one of claims 1 to 5, wherein the melflufen, or salt thereof, is administered simultaneously, sequentially or separately with one or more further therapeutic agent(s).

7. Melflufen, or a salt thereof, for use as claimed in claim 6, wherein the one or more further therapeutic agent(s) is selected from steroids (e.g. prednisone and dexamethasone), IMiDs (e.g. thalidomide, lenalidomide and pomalidomide), PIs (e.g. bortezomib, carfilzomib, and ixazomib), histone deacetylase (HDAC) inhibitors (e.g. panobinostat), conventional chemotherapy (e.g. melphalan, cyclophosphamide, doxorubicin, bendamustine), anti-CD38 antibodies (daratumumab) and anti-SLAMF7 antibodies (elotuzumab); and preferably the one or more further therapeutic agent(s) selected from dexamethasone, daratumumab and bortezomib; or selected from antibodies against the B-cell maturation antigen (e.g. belantamab), inhibitors of nuclear export (e.g. selinexor) and autologous chimeric antigen receptor (CAR) T-cell therapy directed against the B-cell maturation antigen (e.g. ciltacabtagene), and more preferably the further therapeutic agent is dexamethasone.

8. A pharmaceutical formulation comprising melflufen, or a salt thereof, for use as defined in any one of claims 1 to 7.

9. A method for the treatment or prophylaxis of multiple myeloma, comprising the step of administering melflufen, or a salt thereof, to a patient having multiple myeloma who
has not received a stem cell transplant; or
has received a stem cell transplant that was at least 5 years ago; or
is 75 years old or older; or
has not received a stem cell transplant and is 75 years old or older, or has received a stem cell transplant that was at least 5 years ago and is 75 years old or older; or
has received a stem cell transplant and the disease subsequently progressed at least 36 months after the transplant.

10. The method of claim 9, wherein the patient is additionally administered simultaneously, sequentially or separately from melflufen, or a salt thereof, one or more further therapeutic agent(s) which is selected from steroids (e.g. prednisone and dexamethasone), IMiDs (e.g. thalidomide, lenalidomide and pomalidomide), PIs (e.g. bortezomib, carfilzomib, and ixazomib), histone deacetylase (HDAC) inhibitors (e.g. panobinostat), conventional chemotherapy (e.g. melphalan, cyclophosphamide, doxorubicin, bendamustine), anti-CD38 antibodies (daratumumab) and anti-SLAMF7 antibodies (elotuzumab); and preferably one or more further therapeutic agent(s) selected from dexamethasone, daratumumab and bortezomib; or selected from antibodies against the B-cell maturation antigen (e.g. belantamab), inhibitors of nuclear export (e.g. selinexor) and autologous chimeric antigen receptor (CAR) T-cell therapy directed against the B-cell maturation antigen (e.g. ciltacabtagene), and more preferably the further therapeutic agent is dexamethasone.

11. The method of claim 9 or 10, wherein the method comprises determining if the patient has received for a stem cell transplant and if the patient has not received a stem cell transplant, administering melflufen, or a salt thereof, to the patient; or
determining if the patient has received a stem cell transplant that was at least 5 years ago and if the patient has received a stem cell transplant that was at least 5 years ago, administering melflufen, or a salt thereof, to the patient; and/or
determining if the patient is 75 years old or older, and if the patient is 75 years old or older, administering melflufen, or a salt thereof, to the patient; and/or
determining if the patient has received a stem cell transplant and the disease subsequently progressed at least 36 months after the transplant, and if the patient has received a stem cell transplant and the disease subsequently progressed at least 36 months after the transplant, administering melflufen, or a salt thereof, to the patient.

12. The method of claim 9, 10 or 11, wherein the method comprises determining if the patient is suitable for a stem cell transplant and if the patient is not suitable for a stem cell transplant, administering melflufen, or a salt thereof, to the patient.

13. The use of melflufen, or a salt thereof, for the manufacture of a medicament for the treatment of multiple myeloma in a patient having multiple myeloma who
has not received a stem cell transplant; or
has received a stem cell transplant that was at least 5 years ago; or
is 75 years old or older; or
has not received a stem cell transplant and is 75 years old or older, or has received a stem cell transplant that was at least 5 years ago and is 75 years old or older; or
has received a stem cell transplant and the disease subsequently progressed at least 36 months after the transplant.

14. A kit comprising melflufen and one or more further therapeutic agent(s) selected from steroids (e.g. prednisone and dexamethasone), IMiDs (e.g. thalidomide, lenalidomide and pomalidomide), PIs (e.g. bortezomib, carfilzomib, and ixazomib), histone deacetylase (HDAC) inhibitors (e.g. panobinostat), conventional chemotherapy (e.g. melphalan, cyclophosphamide, doxorubicin, bendamustine), anti-CD38 antibodies (daratumumab) and anti-SLAMF7 antibodies (elotuzumab); or selected from antibodies against the B-cell maturation antigen (belantamab), inhibitors of nuclear export (selinexor) and autologous chimeric antigen receptor (CAR) T-cell therapy directed against the B-cell maturation antigen (ciltacabtagene), and preferably the one or more further therapeutic agent(s) selected from dexamethasone, daratumumab and bortezomib; and more preferably the further therapeutic agent is dexamethasone;
for use in the treatment or prophylaxis of multiple myeloma in a patient as defined in claim 1.

15. The melflufen, or a salt thereof, for use as claimed in any one of claims 1 to 7, the pharmaceutical formulation as claimed in claim 8, the method as claimed in claims 9 to 12, the use as claimed in claim 13, or the kit as claimed in claim 14, wherein the multiple myeloma patient:
has received at least 2 prior lines of therapy for multiple myeloma, for example at least 2 prior lines of therapy including lenalidomide and a protease inhibitor, either sequentially or as part of a combined treatment regimen; and/or
is refractory (for example relapsed and refractory, or refractory) to the last line of therapy and/or to lenalidomide administered within 18 months prior to the treatment; and/or
is refractory (for example relapsed and refractory, or refractory) to at least an alyklator; and/or
is refractory (for example relapsed and refractory, or refractory) to at least an anti-CD38 antibody; and/or
is refractory (for example relapsed and refractory, or refractory) to at least an immunomodulatory drug (IMiDs); and/or
is refractory (e.g. refractory or relapsed-refractory) to lenalidomide, and in particular refractory (e.g. refractory or relapsed-refractory) to lenalidomide wherein lenalidomide was the last treatment that the patient received for multiple myeloma; and/or
is refractory to one or more (for example 1, 2, 3, 4 or 5 or more) drug from two of more classes of drugs selected from protease inhibitors (PIs), immunomodulatory drugs (IMiDs), alkylators and anti-CD38 antibody; and/or
is refractory (e.g. refractory or relapsed-refractory) to at least lenalidomide and 1, 2, 3 or 4 other drugs, for example at least one drug selected from protease inhibitor (PI), immunomodulatory drug (IMiD) alkylators and anti-CD38 antibody (or example, 2, 3 or 4 other drugs including at least one protease inhibitor (PI) and immunomodulatory drug (IMID); and/or
is refractory (e.g. refractory or relapsed-refractory) to at least pomalidomide and/or daratumumab; and/or
has RRMM.

16. The melflufen, or a salt thereof, for use as claimed in any one of claim 1 to 7 or 15, the pharmaceutical formulation as claimed in claim 8 or 15, the method as claimed in claim 9 to 12 or 15, the use as claimed in claim 13 or 15, or the kit as claimed in claim 14 or 15, wherein the multiple myeloma patient:
is at least 65, 70, 75 or 80 years old; and/or
has cardiovascular disease; and/or
has pulmonary disease.

17. The melflufen, or a salt thereof, for use as claimed in any one of claims 1 to 7 or 15 to 16, the pharmaceutical formulation as claimed in claims 8 or 15 to 16, the method as claimed in claims 9 to 12 or 15 to 16, the use as claimed in claims 13 or 15 to 16, or the kit as claimed in claims 14 to 16, wherein the multiple myeloma patient is not suitable for a stem cell transplant.

18. The melflufen, or a salt thereof, for use as claimed in any one of claims 1 to 7 or 15 to 17, the pharmaceutical formulation as claimed in claims 8 or 15 to 17, the method as claimed in claims 9 to 12 or 15 to 17, the use as claimed in claims 13 or 15 to 17, or the kit as claimed in claims 14 to 17, wherein the multiple myeloma patient:
has a median body surface area (BSA) of ≤1.855 m²; and/or
has multiple myeloma with a Revised Multiple Myeloma International Staging System (R-ISS) of ISS grouping of I or II; and/or
is a high risk patients in view of the patient's cytogenetics; and/or
has impaired kidney function (for example, a creatine clearance of less than 60 milliliters per minute (mL/min), or 60 to 90 mL/min; and in particular a creatine clearance of less than 60 mL/min.

19. The melflufen, or a salt thereof, for use as claimed in any one of claims 1 to 7 or 15 to 18, the pharmaceutical formulation as claimed in claims 8 or 15 to 18, the method as claimed in claims 9 to 12 or 15 to 18, the use as claimed in claims 13 or 15 to 18, or the kit as claimed in claims 14 to 18, wherein the multiple myeloma patient:
has RRMM; and/or (preferably and)
is refractory to lenalidomide (for example refractory to lenalidomide (for example ≥10 mg) administered within 18 months prior to treatment); and/or (preferably and)
has received at least 2 previous lines of therapy (for example 2, 3, or 4 previous lines of therapy).

20. The melflufen, or a salt thereof, for use as claimed in any one of claims 1 to 7 or 15 to 19, the pharmaceutical formulation as claimed in claims 8 or 15 to 19, the method as claimed in claims 9 to 12 or 15 to 19, the use as claimed in claims 13 or 15 to 19, or the kit as claimed in claims 14 to 19, wherein the multiple myeloma patient has not received a stem cell transplant.
